(19) United States
(12) Patent Application Publication
(10) Pub. No.: US 2018/0140273 A1
(43) Pub. Date: May 24, 2018

(54) ULTRASONIC SIGNAL PROCESSING APPARATUS, ULTRASONIC IMAGING APPARATUS USING SAME, AND CONTROL METHOD IN ULTRASONIC SIGNAL PROCESSING APPARATUS

(71) Applicant: HITACHI, LTD., Tokyo (JP)

(72) Inventors: Takahide TERADA, Tokyo (JP); Kenichi KAWABATA, Tokyo (JP); Atsurou SUZUKI, Tokyo (JP); Yushi TSUBOTA, Tokyo (JP); Wenjing WU, Tokyo (JP); Kazuhiro YAMANAKA, Tokyo (JP)

(73) Assignee: HITACHI, LTD., Tokyo (JP)

(21) Appl. No.: 15/819,856

(22) Filed: Nov. 21, 2017

(30) Foreign Application Priority Data
Nov. 22, 2016 (JP) 2016-226402

(51) Int. Cl. A61B 8/00; G01S 15/89; A61B 8/08

(52) U.S. Cl. CPC A61B 8/4281; G01S 15/8922; A61B 8/4483; A61B 8/406; A61B 8/0825

(57) ABSTRACT

There is provided an ultrasonic signal processing apparatus in which the amount of an ultrasonic propagation material in a measurement unit that stores a transducer array is reduced while the transducer array and a target are separated from each other, and thus maintenance of the ultrasonic propagation material is easily managed.

The ultrasonic signal processing apparatus includes a water tank having an opening portion, a measurement unit, and a moving unit. The measurement unit which is disposed on an outside of the water tank, includes a transducer array that transmits and receives an ultrasonic signal, and a space which is filled with a material for propagating the ultrasonic signal, and measures the ultrasonic signal. The moving unit is connected to the measurement unit, and moves the measurement unit between the opening portion and the bottom portion of the water tank, along a side wall thereof.

### TECHNICAL FIELD

The present invention relates to an ultrasonic signal processing apparatus, an ultrasonic imaging apparatus using the same, and a control method in the ultrasonic signal processing apparatus.

### BACKGROUND ART

As an ultrasonic computed tomography (CT) apparatus, there is an ultrasonic imaging apparatus as follows. The ultrasonic imaging apparatus transmits an ultrasonic wave to an inside of a target disposed in an ultrasonic propagation material (water and the like) which is a medium for propagating an ultrasonic wave. The apparatus receives an ultrasonic wave passing through the inside of the target on each of a plurality of paths, and measures a propagation time and the like from the transmission of the ultrasonic wave until being received. The apparatus calculates distribution (acoustic characteristic distribution) of physical property values (sound speed, attenuation of ultrasonic wave, or the like) which reflect acoustic characteristics of the target, based on the propagation time, the length (propagation distance) of propagation path, and the like, and thus generates a tomographic image of the target. That is, the ultrasonic CT apparatus obtains a tomographic image of a target regarding predetermined physical property values, by using an ultrasonic tomography method.

PTL 1 discloses an ultrasonic CT apparatus having a configuration in which an oil tank that encircles a water tank in which a breast is put is disposed, and a ring-like transducer array is disposed in the oil tank. PTL 1 discloses that the transducer array is movable up and down in the oil tank.

### CITATION LIST

### Patent Literature

PTL 1: U.S. Pat. No. 5,305,752

### SUMMARY OF INVENTION

### Technical Problem

The temperature of an ultrasonic propagation material causes a propagation speed of an ultrasonic wave to be changed. Thus, in a case where temperature distribution of an ultrasonic propagation material is not sufficiently uniform, a propagation time of an ultrasonic wave changes and thus it is not possible to calculate acoustic characteristic distribution of a target with high accuracy. In addition, an ultrasonic wave transmitted from a transducer array is scattered by bubbles in the ultrasonic propagation material. Thus, in a case where it is not possible to sufficiently remove the bubbles in the ultrasonic propagation material, a propagation path of an ultrasonic wave changes, and thus it is not possible to calculate acoustic characteristic distribution of the target with high accuracy.

As described above, since the ultrasonic propagation material much largely influences propagation of an ultrasonic wave, special managements regarding material temperature adjustment for holding temperature distribution to be uniform, a degassing treatment of removing bubbles in the ultrasonic propagation material, and the like are required. Therefore, in an ultrasonic imaging apparatus for medical purposes (for example, for breast cancer screening), the amount of an ultrasonic propagation material is preferably small from a viewpoint of, for example, maintenance management of the ultrasonic propagation material.

As in PTL 1, if a target and a transducer array are disposed in tanks different from each other and are separated from each other, an ultrasonic propagation material with which the outer tank in which the transducer array is stored is filled is required in addition to an ultrasonic propagation material with which the inner tank into which the target is put is filled. Since the transducer array moves up and down in the outer tank, a large amount of the ultrasonic propagation material is required for the outer tank, and thus the amount of the ultrasonic propagation material required by the ultrasonic imaging apparatus is more increased.

An object of the present invention is to provide an ultrasonic signal processing apparatus in which the amount of an ultrasonic propagation material in a measurement unit that stores a transducer array is reduced while the transducer array and a target are separated from each other, and thus maintenance of the ultrasonic propagation material is easily managed.

### Solution to Problem

To solve the above problem, according to the present invention, there is provided an ultrasonic signal processing apparatus as follows. That is, the ultrasonic signal processing apparatus includes a water tank having an opening portion, a measurement unit, and a moving unit. The measurement unit includes a transducer array that transmits and receives an ultrasonic signal, and a space which is filled with a material for propagating the ultrasonic signal, is disposed on the outside of the water tank, and measures the ultrasonic signal. The moving unit is connected to the measurement unit, and moves the measurement unit between the opening portion and the bottom portion of the water tank, along a side wall thereof .

### Advantageous Effects of Invention

According to the present invention, it is possible to provide an ultrasonic signal processing apparatus in which the amount of an ultrasonic propagation material in a measurement unit that stores a transducer array is reduced while the transducer array and a target are separated from each other, and thus maintenance of the ultrasonic propagation material is easily managed.

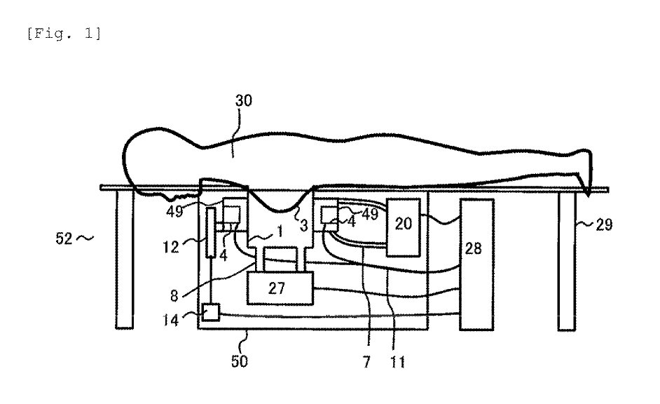

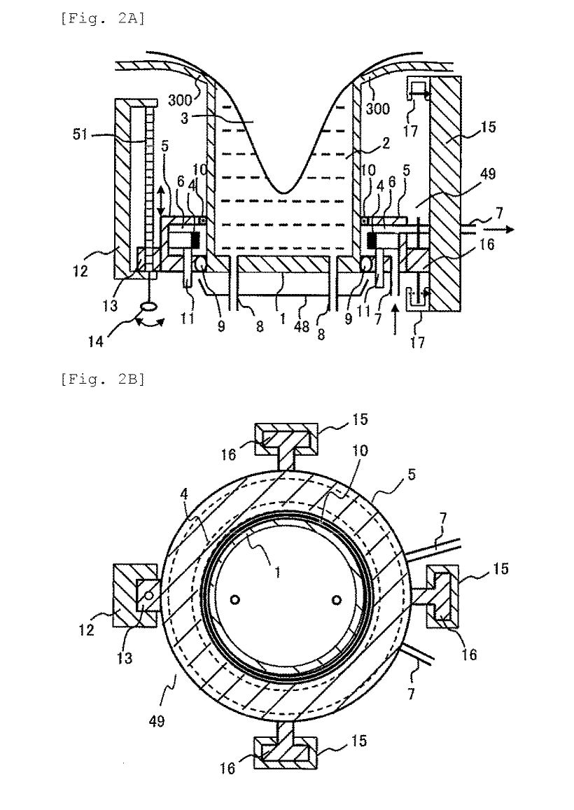

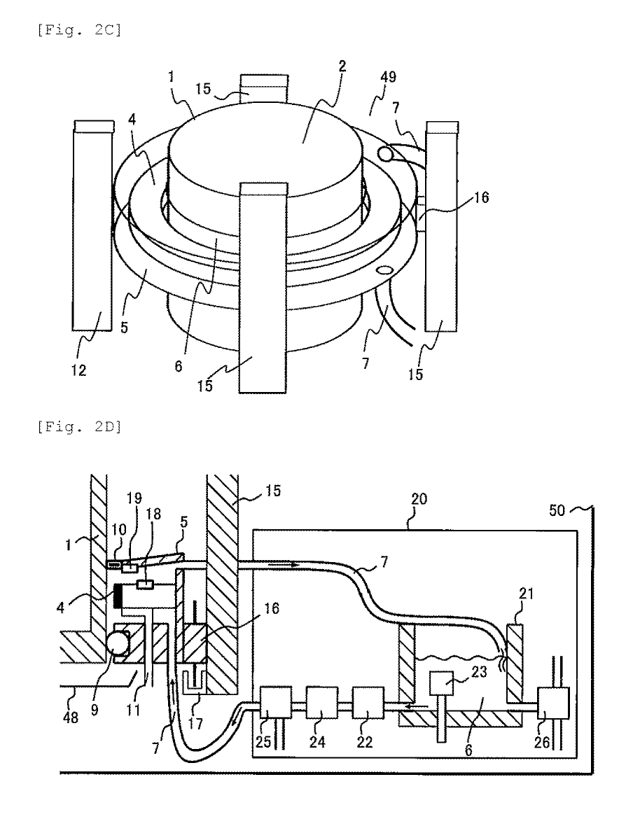

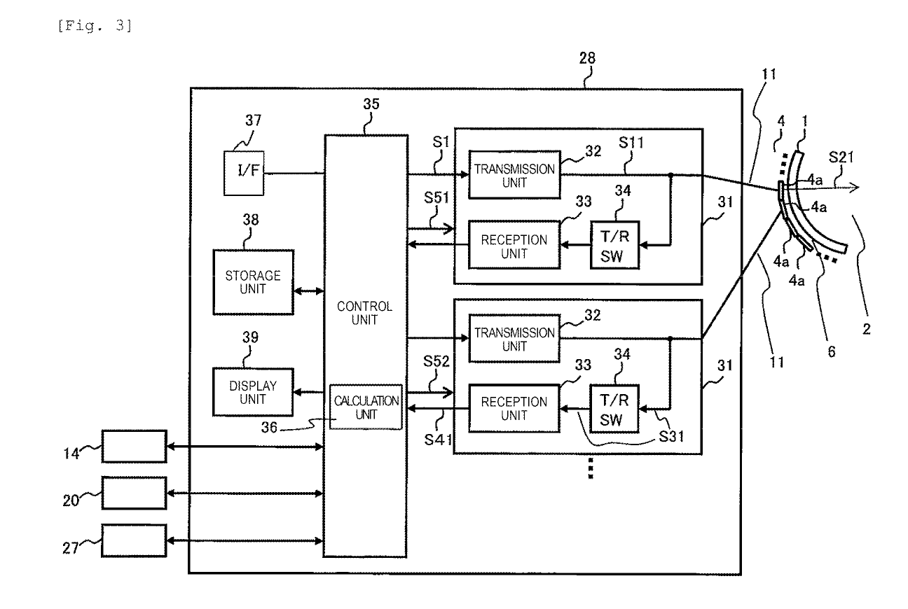

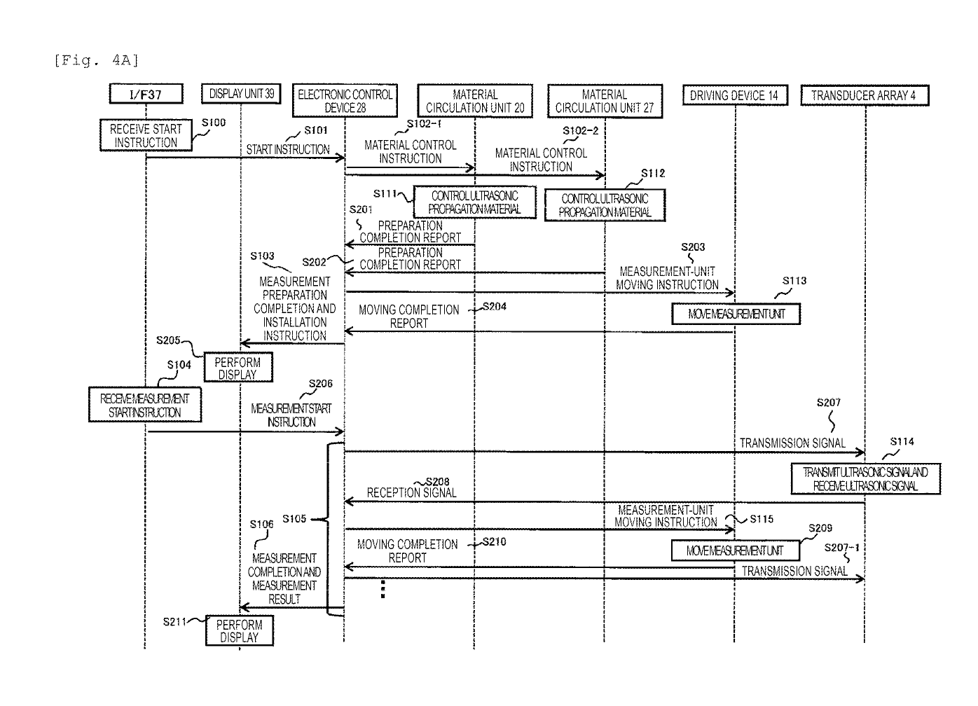

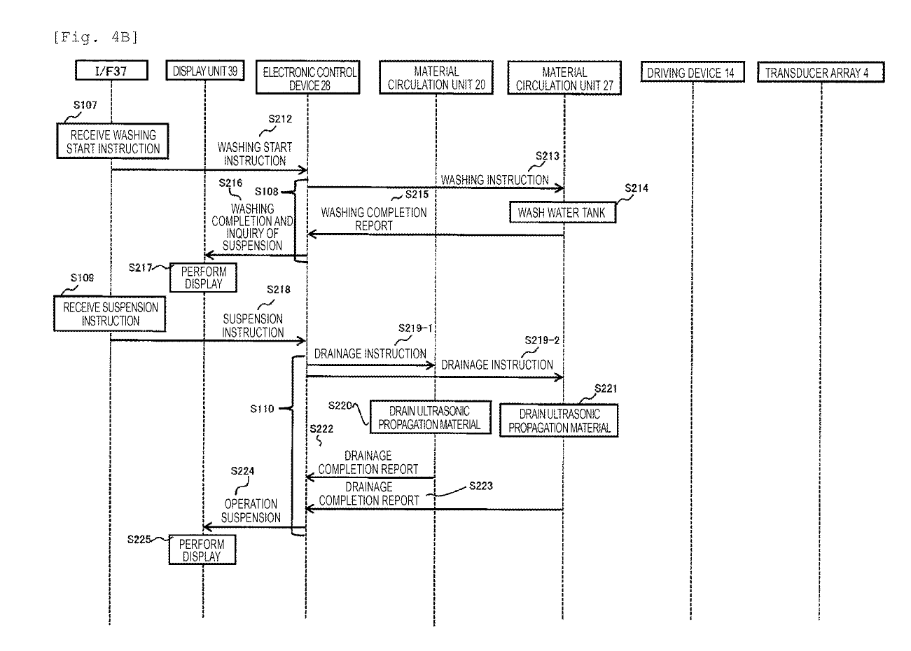

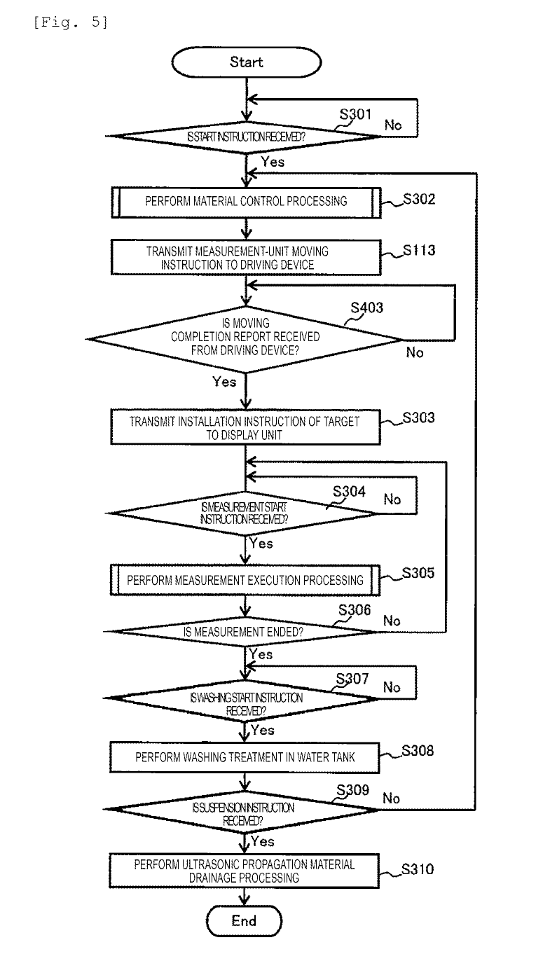

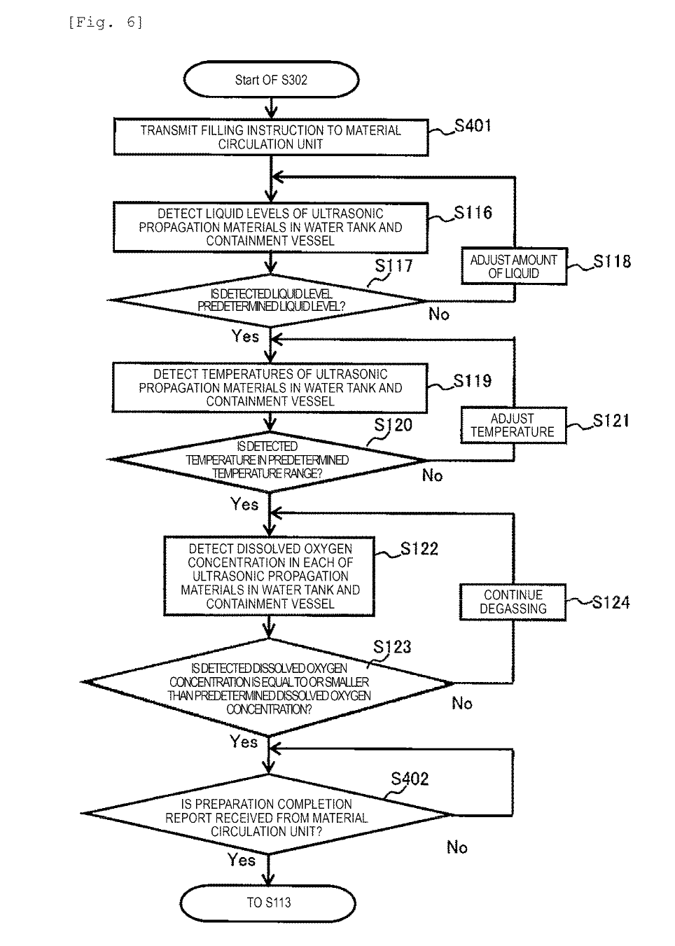

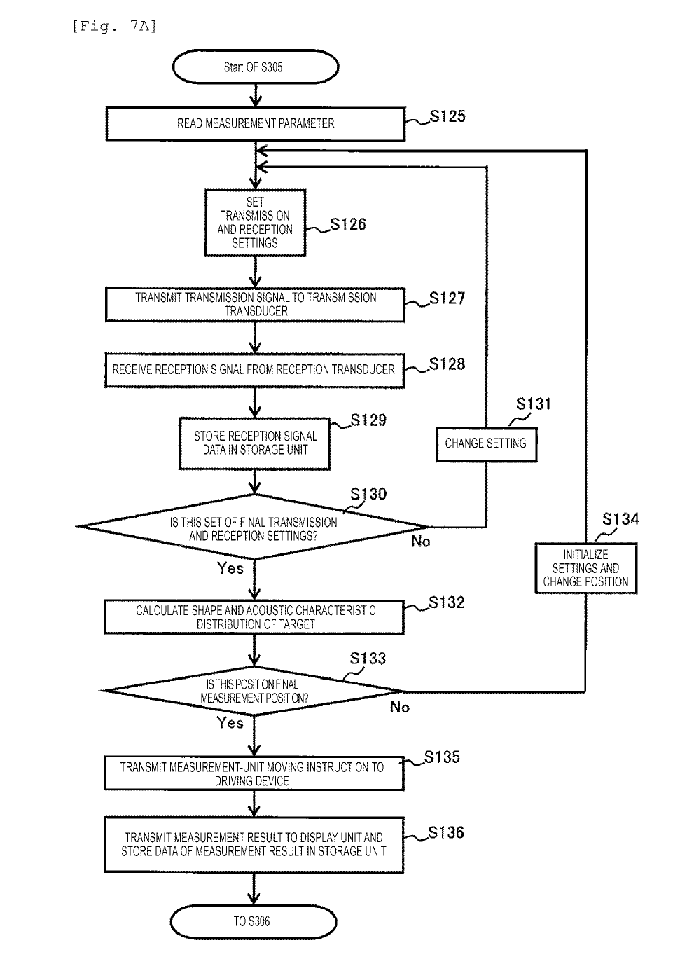

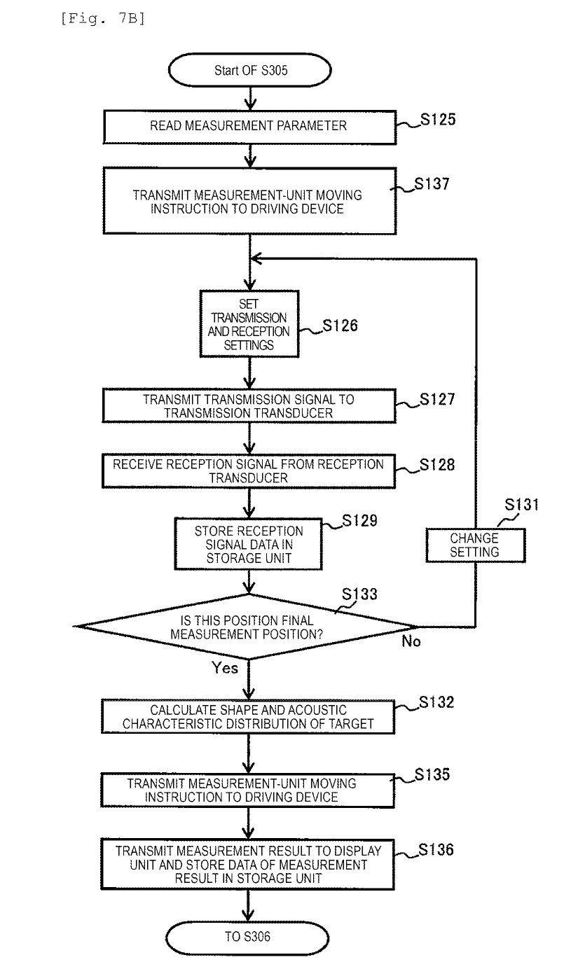

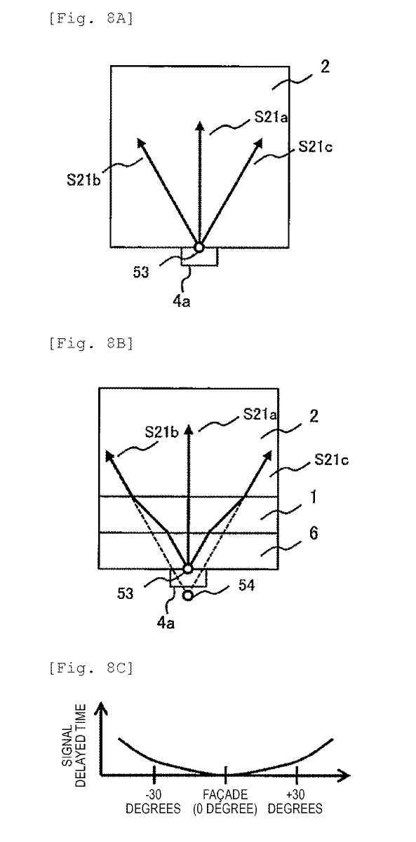

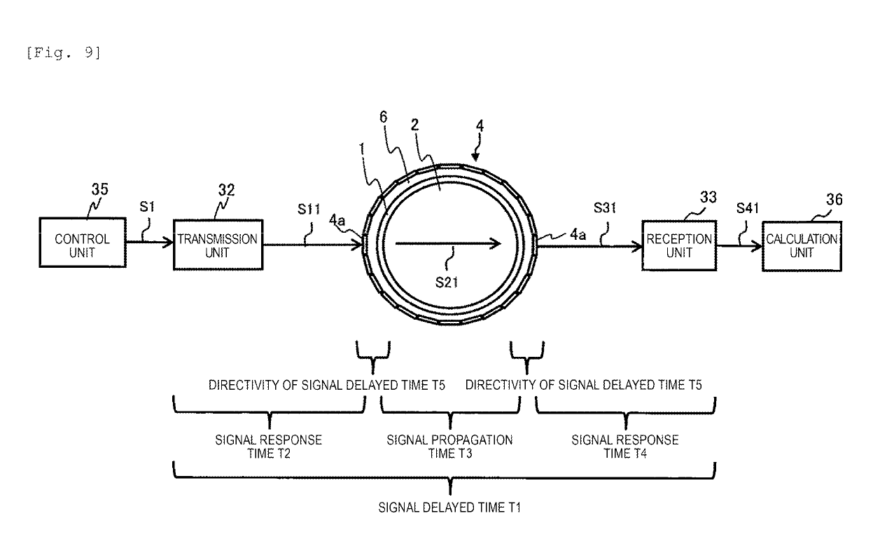

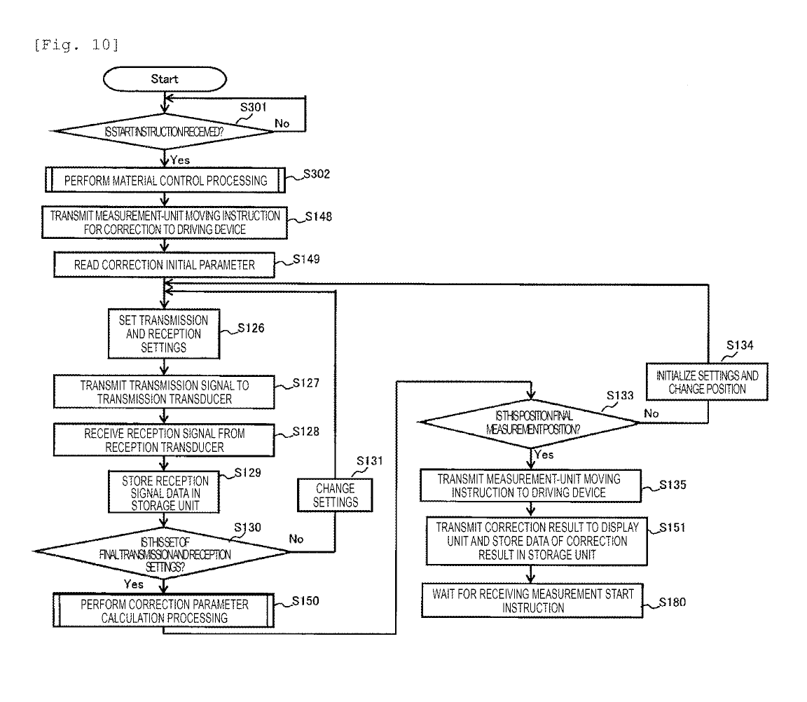

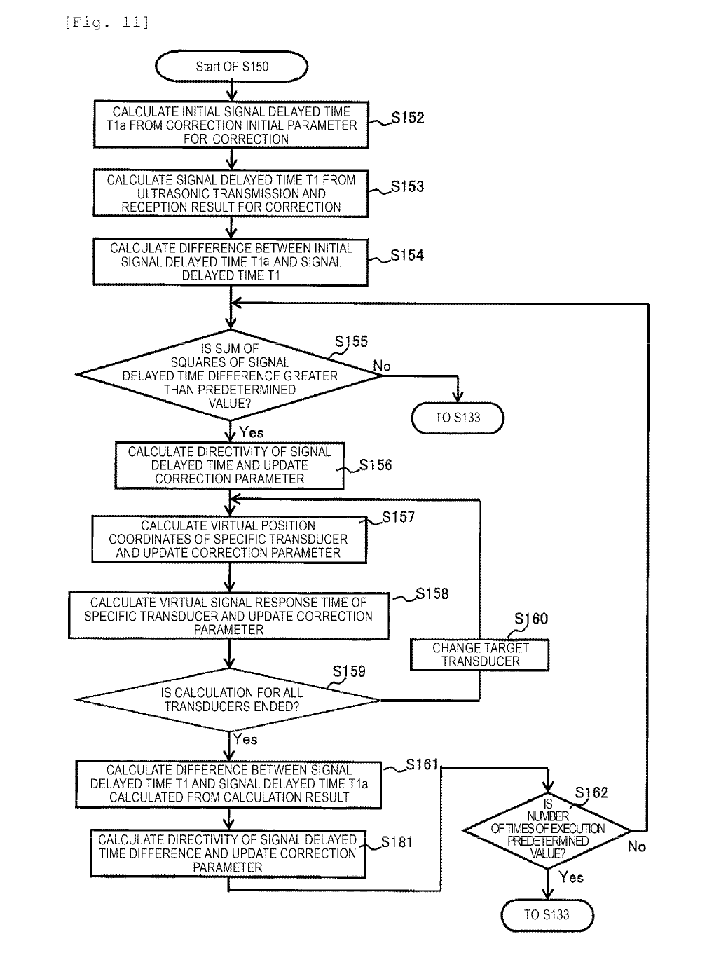

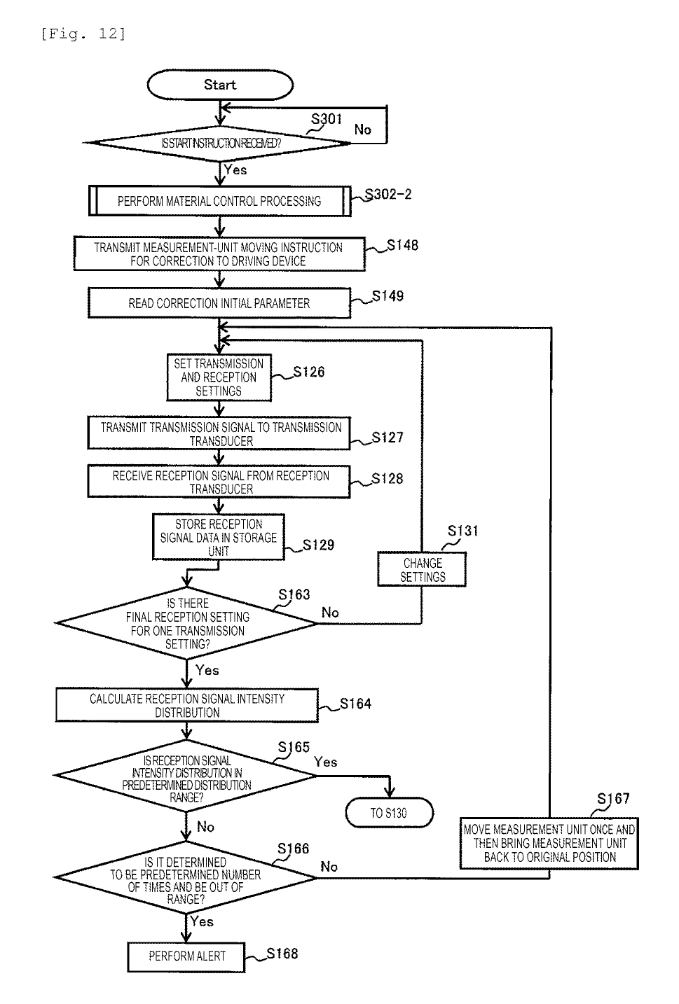

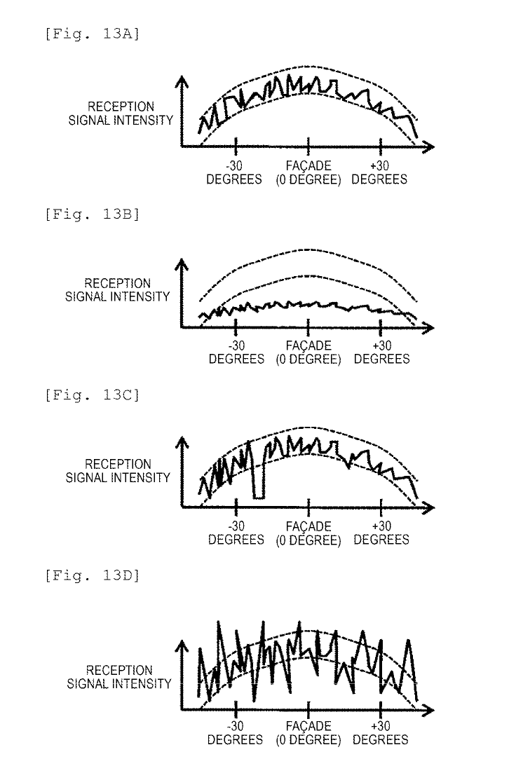

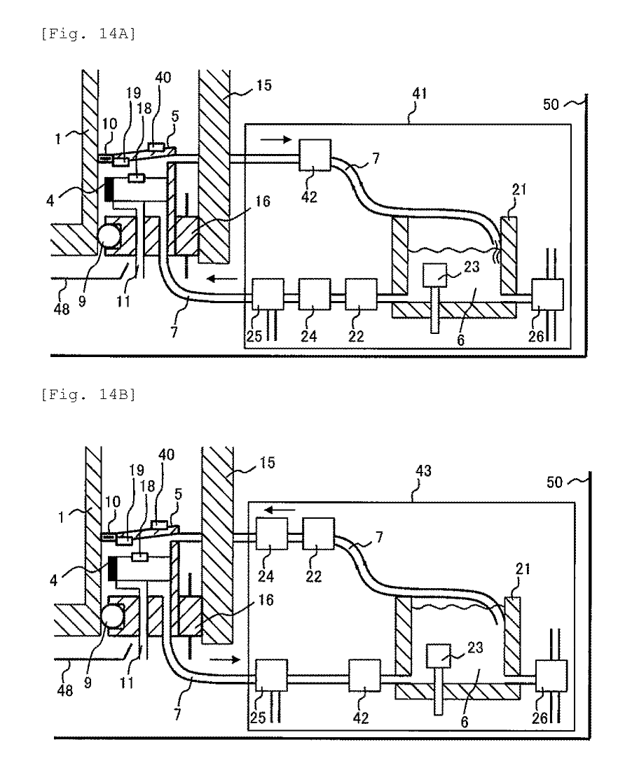

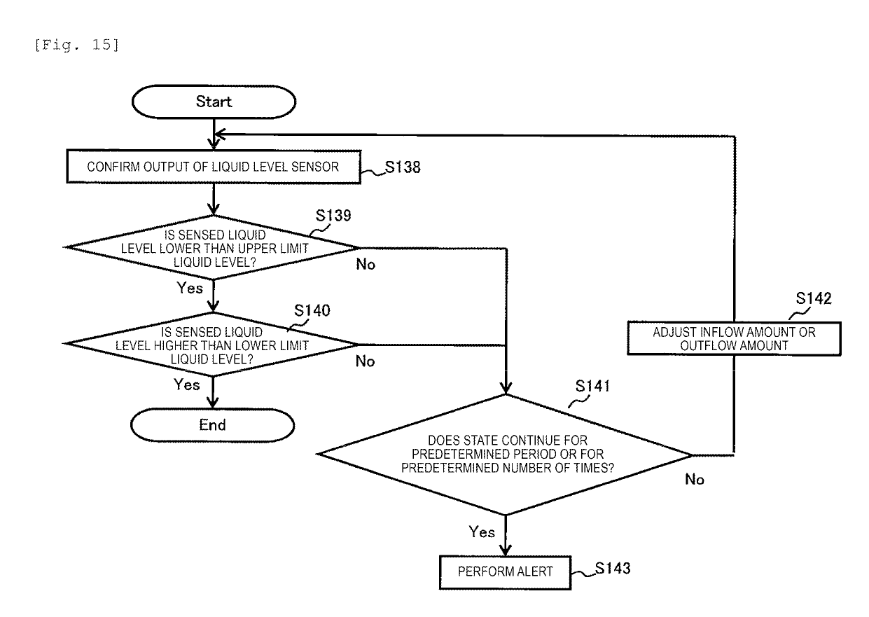

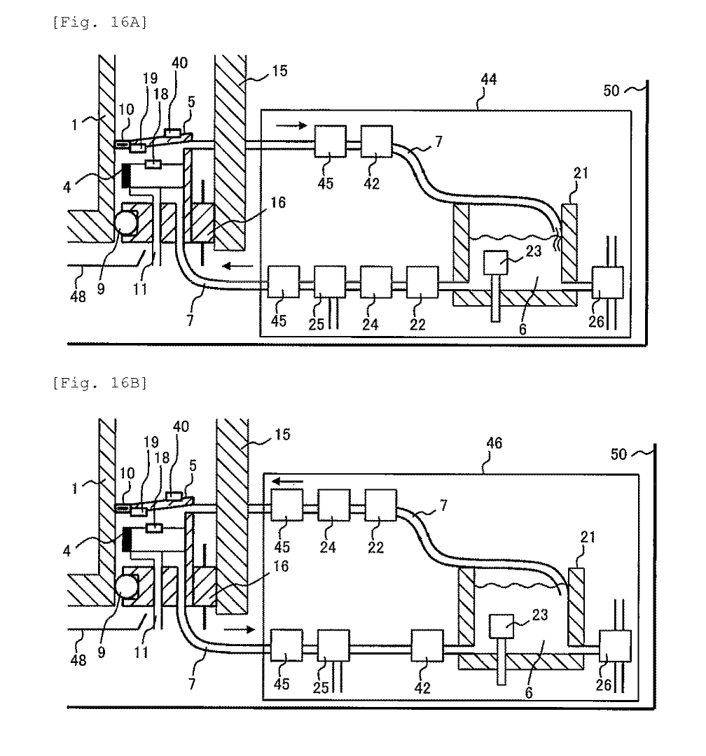

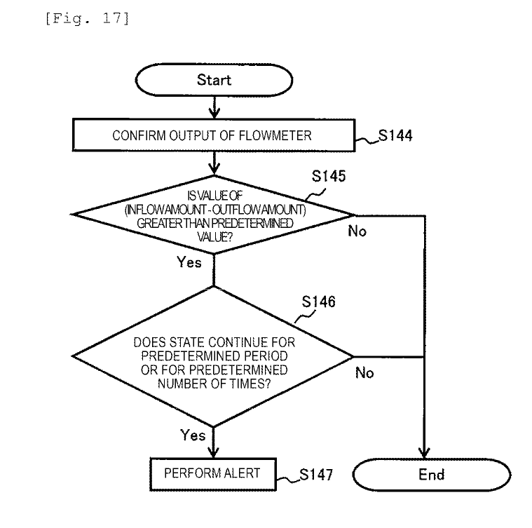

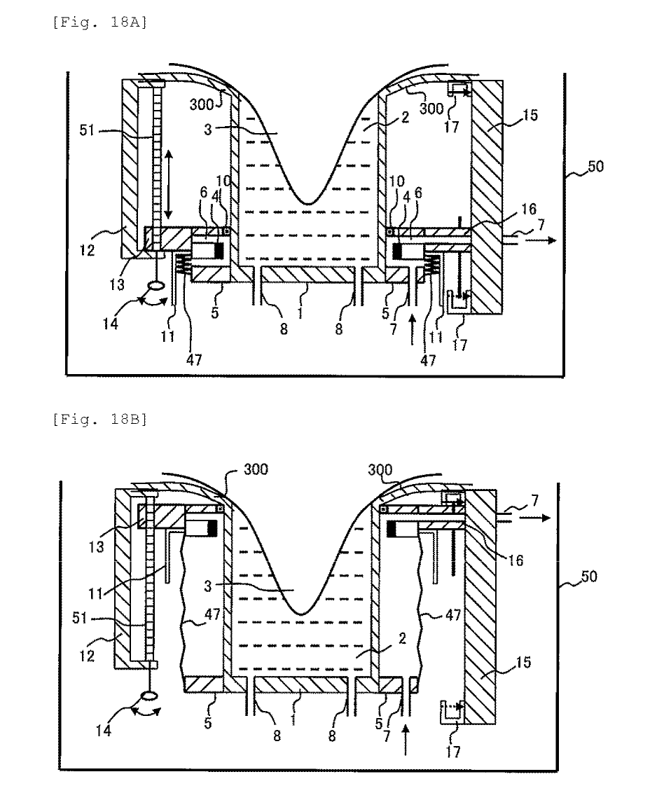

### BRIEF DESCRIPTION OF DRAWINGS

[FIG. 1] FIG. 1 is a diagram illustrating an overall configuration example of an ultrasonic imaging apparatus.

[FIG. 2A] FIG. 2A is a diagram illustrating an example of a peripheral structure of a measurement unit and a water tank.

[FIG. 2B] FIG. 2B is a diagram illustrating an example when the peripheral structure of the measurement unit and the water tank is viewed from the top.

[FIG. 2C] FIG. 2C is a perspective view illustrating an example of the peripheral structure of the measurement unit and the water tank.

[FIG. 2D] FIG. 2D is a diagram illustrating an example of the measurement unit and a material circulation unit that circulates an ultrasonic propagation material in the measurement unit.

[FIG. 3] FIG. 3 is a block diagram illustrating an example of an electronic control device of the ultrasonic imaging apparatus.

[FIG. 4A] FIG. 4A is a sequence diagram illustrating an operation example of the ultrasonic imaging apparatus.

[FIG. 4B] FIG. 4B is a sequence diagram illustrating an operation example of the ultrasonic imaging apparatus.

[FIG. 5] FIG. 5 is a flowchart illustrating an example of an overall flow of an operation of a control unit.

[FIG. 6] FIG. 6 is a flowchart illustrating an example of details of material control processing by the control unit.

[FIG. 7A] FIG. 7A is a flowchart illustrating an example of details of measurement execution processing by the control unit.

[FIG. 7B] FIG. 7B is a flowchart illustrating a modification example of details of the measurement execution processing by the control unit.

[FIG. 8A] FIG. 8A is a diagram illustrating an ultrasonic wave propagation path in a case where a transducer array is installed in the water tank

[FIG. 8B] FIG. 8B is a diagram illustrating an ultrasonic wave propagation path through the water tank, the ultrasonic propagation material in water tank, and an ultrasonic propagation material in a containment vessel.

[FIG. 8C] FIG. 8C is a diagram illustrating directivity of a signal delayed time of an ultrasonic signal.

[FIG. 9] FIG. 9 is a diagram illustrating a signal propagation path in an ultrasonic imaging apparatus.

[FIG. 10] FIG. 10 is a flowchart illustrating an example of a correction parameter calculation operation of the measurement unit which includes the ultrasonic propagation material between a wall of the water tank and the containment vessel, the correction parameter calculation operation being performed by the control unit.

[FIG. 11] FIG. 11 is a flowchart illustrating an example of details of correction parameter calculation processing by the control unit.

[FIG. 12] FIG. 12 is a flowchart illustrating a modification example of the correction parameter calculation operation of the measurement unit which includes the wall of the water tank and the ultrasonic propagation material in the containment vessel, the correction parameter calculation operation being performed by the control unit.

[FIG. 13A] FIG. 13A is an example of predetermined distribution in S165 in FIG. 12.

[FIG. 13B] FIG. 13B is another example of predetermined distribution in S165 in FIG. 12.

[FIG. 13C] FIG. 13C is still another example of predetermined distribution in S165 in FIG. 12.

[FIG. 13D] FIG. 13D is still another example of predetermined distribution in S165 in FIG. 12.

[FIG. 14A] FIG. 14A is a diagram illustrating a modification example of the measurement unit and the material circulation unit that circulates the ultrasonic propagation material in the measurement unit.

[FIG. 14B] FIG. 14B is a diagram illustrating a modification example of the measurement unit and the material circulation unit that circulates the ultrasonic propagation material in the measurement unit.

[FIG. 15] FIG. 15 is a flowchart illustrating an operation example of the control unit, in which control is performed so as to cause the ultrasonic propagation material which remains on an upper surface of the containment vessel not to overflow, in the configuration illustrated in FIG. 14.

[FIG. 16A] FIG. 16A is a diagram illustrating another modification example of the measurement unit and the material circulation unit that circulates the ultrasonic propagation material in the measurement unit.

[FIG. 16B] FIG. 16B is a diagram illustrating another modification example of the measurement unit and the material circulation unit that circulates the ultrasonic propagation material in the measurement unit.

[FIG. 17] FIG. 17 is a flowchart illustrating an operation example of the control unit, in which it is detected whether or not liquid leakage of the ultrasonic propagation material occurs in the containment vessel or a hose, in the configuration illustrated in FIG. 16.

[FIG. 18A] FIG. 18A is a diagram illustrating a modification example of the containment vessel.

[FIG. 18B] FIG. 18B is a diagram illustrating a modification example of the containment vessel.

### DESCRIPTION OF EMBODIMENTS

Hereinafter, an embodiment of the present invention will be described with respect to the drawings. In the drawings for describing the embodiment, components having the same function are denoted by the same names and the same reference signs, and descriptions thereof will not be repeated.

FIG. 1 is a diagram illustrating an overall configuration example of an ultrasonic imaging apparatus and is a diagram illustrating an example in which measurement is performed on the breast of a person as a measurement target.

An ultrasonic imaging apparatus (ultrasonic CT apparatus) 52 includes a bed 29 on which a measurement subject 30 rides, a main apparatus (ultrasonic signal processing apparatus) 50, and an electronic control device 28. The main apparatus 50 transmits and receives an ultrasonic wave (ultrasonic signal) so as to perform ultrasonic wave imaging on a measurement target. The electronic control device 28 controls the main apparatus 50. The main apparatus 50 includes a water tank 1, a measurement unit 49, and a stage 12. In the water tank 1, an opening portion is provided, and a target 3 is inserted into the opening portion. The measurement unit 49 measures an ultrasonic signal (transmits and receives an ultrasonic wave). The stage 12 is a support unit that supports the measurement unit 49.

The water tank 1 is filled with an ultrasonic propagation material which is a material for propagating an ultrasonic wave. The measurement unit 49 includes a transducer array 4 configured to transmit and receive an ultrasonic wave. The measurement unit 49 is filled with an ultrasonic propagation material which is different from the ultrasonic propagation material in the water tank 1.

The main apparatus 50 further includes a material circulation unit 20, a driving device 14, and a material circulation unit 27. The material circulation unit 20 circulates the ultrasonic propagation material in the measurement unit 49 through a hose 7. The driving device 14 moves the measurement unit 49 (vertically moves) along the wall surface (side wall surface) in the water tank 1 on the stage 12. The material circulation unit 27 circulates the ultrasonic propagation material in the water tank 1 through a hose 8. In this example, the opening portion side of the water tank 1 is referred to as an upper portion, and the bottom side of the water tank 1 is referred to as a lower portion. Movement of the measurement unit 49 moving between the opening portion and the bottom portion is referred to as vertical movement.

The ultrasonic propagation material in the water tank 1 is circulated by the material circulation unit 27 and the temperature and the like thereof are managed to be in a state which is suitable for ultrasonic wave imaging. The ultrasonic propagation material in the measurement unit 49 is circulated by the material circulation unit 20 and the temperature and the like thereof are managed to be in a state which is suitable for ultrasonic wave imaging.

The electronic control device 28 is connected to each unit of the main apparatus 50 via each signal wiring 11, and controls the units. Specifically, the electronic control device 28 controls the material circulation unit 20, the material circulation unit 27, and the driving device 14. Thus, the electronic control device 28 outputs a transmission signal for transmission of an ultrasonic wave to the transducer array 4 and receives a reception signal which is input as a result of receiving the ultrasonic wave, from the transducer array 4. Practical devices are not required to have a positional relationship illustrated in FIG. 1. Electric wirings are provided between the electronic control device 28, and the material circulation unit 20, the material circulation unit 27, the driving device 14, and the transducer array 4. However, components may be connected in a manner of wireless communication.

As illustrated in FIG. 1, the measurement subject 30 lies his/her face downward on the bed 29, and thus the breast as a target 3 to be measured is inserted into the water tank 1 through the opening Portion. The transducer array 4 in the measurement unit 49 disposed on the outside of the water tank 1 transmits and receives an ultrasonic wave to and from the target 3 in the water tank 1.

FIG. 2 is a diagram illustrating the measurement unit, the water tank, and the material circulation unit configured to circulate the ultrasonic propagation material in the measurement unit. FIG. 2A is a diagram illustrating an example of a peripheral structure of the measurement unit and the water tank. FIG. 2B is a diagram illustrating an example when the peripheral structure of the measurement unit and the water tank is viewed from the upper Portion. FIG. 2C is a perspective view illustrating an example of the peripheral structure of the measurement unit and the water tank. FIG. 2D is a diagram illustrating an example of the measurement unit and the material circulation unit configured to circulate the ultrasonic propagation material in the measurement unit.

As illustrated in FIGS. 2A to 2C, the measurement unit 49 includes the transducer array 4, a containment vessel 5 configured to accommodate the transducer array 4, and an ultrasonic propagation material 6 with which the containment vessel 5 is filled. The transducer array 4 is a piezoelectric element configured to transmit and receive an ultrasonic wave. A transmission and reception surface of an ultrasonic wave is directed toward the water tank 1. The transducer array 4 is not necessarily limited to an annular shape of covering the entirety of the surroundings of the water tank 1 and a portion of the ring may be cut. In order to simplify a manufacturing process, a plurality of arc sub-transducer arrays may be assembled so as to form an annular transducer array 4.

The water tank 1 or the transducer array 4 are not necessarily columnar or annular. The water tank or the transducer array may have a polygonal cylinder shape or a ring shape. A material which is suitable from a viewpoint of propagation characteristics of an ultrasonic wave, manufacturing cost, or the like can be selected as the material of the water tank 1. For example, resins such as polyethylene, ABS, and polyethylene terephthalate are provided. The wall of the water tank 1 is preferably thin from a viewpoint of the propagation characteristics of an ultrasonic wave. The ultrasonic propagation materials 2 and 6 are acoustic matching materials which are liquids in which an ultrasonic wave is easily transmitted, and are liquids (acoustic matching liquids) for matching with acoustic impedance of a propagation path of an ultrasonic wave. A material which is suitable from a viewpoint of, for example, propagation characteristics of an ultrasonic wave, or manufacturing or treatment cost can be selected as the ultrasonic propagation material. For example, water (degassed water) or a physiological salt solution is provided as the ultrasonic propagation material. The ultrasonic propagation material may be sol, gel, or the like. The ultrasonic propagation materials 2 and 6 may be the same materials or may be materials different from each other.

In order to prevent an occurrence of a situation in which the ultrasonic propagation material 6 is leaked out from the containment vessel 5, a liquid-tight structure 9 is provided on a contact surface between the wall of the containment vessel 5 on a lower surface and the wall of the water tank 1. Since the containment vessel 5 moves up and down along the wall surface of the water tank 1, the liquid-tight structure 9 preferably has a structure having low sliding resistance and high liquid tightness. For example, an O-ring, an X-ring, an U packing, or the like made of synthetic rubber is provided.

The following is considered. When the containment vessel 5 moves up and down, a small gap is formed between the liquid-tight structure 9 and the wall surface of the water tank 1, and thus the ultrasonic propagation material 6 is leaked. In particular, when the containment vessel 5 turns to fall after rising up to the top or the containment vessel 5 turns to rise after falling up to the bottom, the liquid-tight structure 9 may be twisted, and thus there is a probability of forming a gap. In a case where the water tank 1 is made of a resin and the wall surface is thin, the water tank is slightly deformed by pressure which is generated by the liquid-tight structure 9, and the liquid-tight property is totally or locally decreased. Thus, there is a probability of the ultrasonic propagation material 6 being easily leaked. A material receiving tray 48 is provided under the liquid-tight mechanism 9. Thus, the leaked ultrasonic propagation material 6 is collected and drained or is brought back into the material circulation unit 20.

A hole structure 10 is provided on a contact surface between the wall of the containment vessel 5 on an upper surface and the wall of the water tank 1. The hole structure 10 is provided in order to escape an air in the containment vessel 5 and prevent an occurrence of a situation in which bubbles adhere to the wall of the water tank 1 when the containment vessel 5 moves up and down. The hole structure 10 is a ventilation structure of causing an air in the containment vessel 5 to pass. As the hole structure 10, a structure in which the ultrasonic propagation material 6 is permeated and which has elasticity for suppressing wear of the wall surface of the water tank 1. For example, a foam structure or a brush structure made of rubber, a resin, urethane, or the like is provided. The ultrasonic propagation material 6 is permeated into the hole structure 10, and thus it is possible to decrease sliding resistance between the hole structure 10 and the water tank 1.

The hole structure 10 is set to be in contact with a liquid surface of the ultrasonic propagation material 6 or the liquid level of the ultrasonic propagation material 6 is set to be higher than the hole structure 10. Thus, it is possible to prevent an occurrence of a situation in which, when the containment vessel 5 moves up and down, the liquid level of the ultrasonic propagation material 6 coming into contact with the wall of the water tank 1 is changed in the containment vessel 5 by surface tension of the ultrasonic propagation material 6 or bubbles adhere to the liquid surface of the ultrasonic propagation material 6 by foreign matters adhering to the wall of the water tank 1, minute scratches, or the like. In addition, when the containment vessel 5 moves up and down, the liquid-tight structure 9 removes foreign matters on the surface, which adhere to the wall of the water tank 1, and thus it is possible to prevent bubbles from being generated.

It is also considered that a structure which is similar to the liquid-tight structure 9 is provided instead of the hole structure 10, and a hole for escaping an air is provided at a place which is different from the wall of the containment vessel 5 on the upper surface. However, in this case, the wall of the containment vessel 5 on the upper surface becomes thick and measurement in the vicinity of the opening portion of the water tank 1 is not possible, and a blind area which is an area of which measurement is not possible is largely formed. Generally, if the diameter of the water tank 1 is set to be about 200 mm, the thickness of the liquid-tight structure 9 provided on the wall of the containment vessel 5 on the upper surface thereof is set to be about 10 mm. This is not suitable in a case where the breast is measured as the target 3 in order to detect a breast cancer. On the contrary, in a case where the hole structure 10 is provided, the thickness of the wall of the containment vessel 5 on the upper surface thereof may be set to be about 1 mm and thin. The reason is because a function of escaping an air in the containment vessel 5, a function of permeating the ultrasonic propagation material 6 into the hole structure 10, or a function of preventing adhering of bubbles by being brought into contact with the wall of the water tank 1 is hardly impaired without depending on the thickness of the hole structure 10. The measurement unit 49 which includes the hole structure 10 can move until the hole structure 10 is brought into contact with an upper wall 300 in the vicinity of the opening portion of the water tank 1.

The signal wiring 11 is drawn outwardly from the inside of the containment vessel 5 in order to input a transmission signal to the transducer array 4 or output a reception signal from the transducer array 4. Since the transducer array 4 and the containment vessel 5 are fixed to each other, the signal wiring 11 and the containment vessel 5 are also fixed to each other. Therefore, liquid tightness between the signal wiring 11 and the containment vessel 5 can be easily realized by burying an adhesive, silicon rubber, or the like in the gap.

The signal wiring 11 on at least an outside of the containment vessel 5 is configured by a flexible printed board or a multi-core coaxial cable having flexibility, so as not to disturb vertical movement of the containment vessel 5. The signal wiring 11 has a sufficient length in consideration of the vertical movement of the containment vessel 5, and is connected to the electronic control device 28 (see FIGS. 1 and 3).

The measurement unit 49 is supported by fitting portions 13 and 16 which are connected (fixed) to the containment vessel 5, the stage 12 which is the support unit, and a guide rail 15, and moves up and down along the wall surface of the water tank 1. The fitting portions 13 and 16 are moving units configured to move (vertically move) the measurement unit 49 between the opening portion and the bottom portion of the water tank 1, along the side wall of the water tank 1. The stage 12 and the fitting portion 13 are connected by, for example, a screw structure 51 which is a vertical movement mechanism of moving the measurement unit 49 up and down. The screw structure 51 is rotated by the driving device 14, and thus a relative positional relationship between the stage 12 and the fitting portion 13 changes. The guide rail 15 is fit with the fitting portion 16 such that the measurement unit 49 is held to be vertical to the wall surface of the water tank 1. If three sets or more of stages 12 and guide rails 15 are provided, the measurement unit 49 can move up and down without being inclined from the water tank 1. If the measurement unit 49 is inclined from the water tank 1, pressure on the liquid-tight structure 9 becomes ununiform, and thus the liquid-tight property is deteriorated.

With a structure of moving the measurement unit 49 up and down, the fitting portions 13 and 16 can be provided in a transverse direction of the containment vessel 5, and a space which is provided at the lower portion of the containment vessel 5 for vertical movement can be reduced. Thus, it is possible to accommodate the water tank 1, the measurement unit 49, the stage 12, the guide rail 15, and the like in a space having a small volume. In addition, in a case where a thermostatic tank is provided in the main apparatus 50 in order to hold states of the water tank 1, the ultrasonic propagation materials 2 and 6, and the like under constant conditions, it is possible to reduce the volume of the thermostatic tank.

The guide rail 15 includes a position detection sensor 17, and detects the position of the fitting portion 16. For example, an infrared sensor is used as the position detection sensor 17 and a protrusion for blocking an infrared ray is provided at the fitting portion 16. If the protrusion of the fitting portion 16 is applied to the position detection sensor 17 installed at the lower portion of the guide rail 15, and an infrared ray is blocked, the position detection sensor 17 detects this situation. Since the position detection sensor 17 detects blocking of an infrared ray, it is detected that the fitting portion 16 reaches the bottom portion, that is, that the measurement unit 49 reaches the bottom portion. Similarly, if the protrusion of the fitting portion 16 is applied to the position detection sensor 17 installed at the upper portion of the guide rail 15, and an infrared ray is blocked, the position detection sensor 17 detects this situation. Thus, the position detection sensor 17 detects that the fitting portion 16 reaches the top portion, that is, that the measurement unit 49 reaches the top portion. The position detection sensor 17 is not limited to an infrared sensor, and may be a sensor by using visible light, an electromagnetic wave, a sound wave, an electric signal, or the like.

The installation place of the position detection sensor 17 is not limited to the guide rail 15. The position detection sensor 17 may be installed on the stage 12, in the main apparatus 50, the thermostatic tank, or at another fixable place. A protrusion shape or the like corresponding to any place of the measurement unit 49 may be disposed in accordance with the installation place of the position detection sensor 17. However, the position detection sensor 17 is not installed at a place at which measurement of the target 3 is hindered.

The water tank 1 is joined to the material circulation unit 27 through a hose 8. The containment vessel 5 is joined to the material circulation unit 20 through a hose 7. The hose 7 has a length which is sufficient for causing the vertical movement and measurement accuracy of the measurement unit 49 not to be restricted.

Next, the material circulation unit 20 will be described. As illustrated in FIG. 2D, the material circulation unit 20 includes a material reservoir (material tank) 21, a pump 22, a temperature control device 23, a degassing device 24, a draining device 25, and a feeding and draining device 26. The material circulation unit circulates the ultrasonic propagation material 6 and appropriately manages the temperature and the like of the ultrasonic propagation material 6.

The ultrasonic propagation material 6 is circulated between the containment vessel 5 and the material circulation unit 20 by pressure of the pump 22. The sufficient amount of the ultrasonic propagation material 6 is stored in the material reservoir 21 by the feeding and draining device 26. In a case where the amount of the ultrasonic propagation material 6 is reduced, the ultrasonic propagation material 6 is replenished by the feeding and draining device 26. In order to manage ultrasonic characteristics of the ultrasonic propagation material 6 to be in a desired range, the temperature control device 23 controls the temperature of the ultrasonic propagation material 6 and the degassing device 24 performs degassing by removing a gas (air and the like) included in the ultrasonic propagation material 6.

In a case where the target 3 is a human body, stable measurement is possible if the temperature is controlled to be about 37 degrees which is close to the body temperature. Thus, the temperature control device 23 is realized by, for example, a heater. A heat insulating material may be wound around the hose 7 so as not to change the temperature when the ultrasonic propagation material passes in the hose 7. A thermostatic tank (not illustrated) maybe installed to surround the measurement unit 49, the hose 7, and the material circulation unit 20 through which the ultrasonic propagation material 6 is circulated, and the internal temperature may be set to be substantially equal to a desired temperature of the ultrasonic propagation material 6.

The degassing device 24 is configured by, for example, a filter and a vacuum pump. The filter is configured by a hollow fiber membrane. Since the ultrasonic propagation material 6 passes in the hollow fiber membrane and an outside of the hollow fiber membrane is decompressed by a vacuum pump, a gas or bubbles dissolved in the ultrasonic propagation material 6 are moved to the outside of the hollow fiber membrane. When the ultrasonic propagation material is drained, the ultrasonic propagation material 6 in the containment vessel 5 and the ultrasonic propagation material 6 in the material reservoir 21 are drained by the draining device 25 and the feeding and draining device 26.

Although not illustrated, the ultrasonic propagation material 2 in the water tank 1 is also similarly joined to the material circulation unit 27 (see FIG. 1) through the hose 8. The material circulation unit 27 circulates the ultrasonic propagation material 2 and appropriately manages the temperature and the like of the ultrasonic propagation material 2.

The temperature and the liquid level of the ultrasonic propagation material 6 in the containment vessel 5 are managed by a temperature sensor 18 and a liquid level sensor 19. The temperature sensor 18 detects the temperature of the ultrasonic propagation material 6. Preferably, the temperature sensor 18 is disposed at a position which is close to an ultrasonic transmission and reception surface of the transducer array 4, in a range without an influence on measurement of the target 3. The liquid level sensor 19 detects the liquid level of the ultrasonic propagation material 6, that is, detects whether or not the containment vessel 5 is filled with the ultrasonic propagation material 6 having an amount as much as not influencing ultrasonic transmission and reception characteristics of the transducer array 4.

As described above, the liquid level of the ultrasonic propagation material 6 is set to be the height which is equal to or higher than the bottom surface of the hole structure 10. If the ultrasonic propagation material 6 flows in from the hose 7 connected to the lower portion of the containment vessel 5, and the ultrasonic propagation material 6 flows from the hose 7 connected to the upper portion of the containment vessel 5 out to the material reservoir 21, the height of the hose 7 connected to the upper portion of the containment vessel 5 corresponds to the height of the liquid level of the ultrasonic propagation material 6 in the containment vessel 5. If the wall of the containment vessel 5 on the upper surface thereof has a configuration in which the height of the wall thereof is reduced as the wall thereof is closer to the water tank 1 and the height of the wall of the containment vessel 5 on the upper surface thereof becomes higher as the wall thereof is farther from the water tank 1 (closer to the hose 7) , an effect of preventing an occurrence of a situation in which the hole structure 10 is submerged in the ultrasonic propagation material 6 or bubbles adhere to the wall of the water tank 1 by the hole structure 10 is easily obtained.

With the above-described configuration, the transducer array 4 is disposed on the outside of the water tank 1, and the measurement unit 49 which includes the transducer array 4 and the ultrasonic propagation material 6 moves up and down along the side wall of the water tank 1. Accordingly, it is possible to three-dimensionally measure a target. The state of the ultrasonic propagation material 6 around the transducer array 4 is suitably managed and thus measurement with high accuracy is possible. The wall of the containment vessel 5 on the upper surface is set to be thin, and thus it is possible to perform measurement up to a position close to the opening portion of the water tank 1, that is, to measure acoustic characteristics of the target 3 in a wide range. With the hole structure 10, forming of bubbles by vertical movement of the measurement unit 49 is suppressed, and thus it is possible to process the ultrasonic propagation material 6 in the containment vessel 5 with higher accuracy. Accordingly, measurement with high accuracy is possible. The support for moving the transducer array 4 up and down may have a shape which protrudes from the bottom surface of the containment vessel 5. However, since the stage 12 and the guide rail 15 are arranged in a transverse direction of the containment vessel 5, an effect in that a large space at the lower portion of the containment vessel 5 is not required and it is possible to reduce the volume of the main apparatus 50 is exhibited in addition to the above-described effect. In a case where a thermostatic tank is provided in the main apparatus 50, it is possible to manage the temperature state of the ultrasonic propagation material 6 by the thermostatic tank with higher accuracy, and thus measurement with high accuracy is possible.

FIG. 3 is a block diagram illustrating an example of the electronic control device in the ultrasonic imaging apparatus.

The electronic control device 28 includes transmission and reception units 31, a control unit 35, an interface (I/F) 37, a storage unit 38, and a display unit 39. The transmission and reception units 31 are respectively connected to transducers 4a in the transducer array 4 by the signal wirings 11.

The transmission and reception unit 31 includes a transmission unit 32, a reception unit 33, and a transmission and reception switch (T/R SW) 34 that performs switching between transmission and reception. The transmission and reception unit 31 transmits and receives an ultrasonic wave through the transducer array 4. One transmission and reception unit 31 is connected to one transducer 4a. Each of the transmission and reception units 31 may independently output a transmission signal to the corresponding transducer 4a. In a case where many transducers 4a are provided and all transducers 4a are not used for one time (ultrasonic signal is not transmitted and received), a connection relationship between the transducer 4a and the transmission and reception unit 31 may be switched by an analog switch (not illustrated) or the like, and thus only a transducer 4a to be used may be connected to the transmission and reception unit 31. On and OFF of the analog switch may be switched by the control unit 35.

The control unit 35 controls the main apparatus 50 and the units in the electronic control device 28. The control unit 35 includes a calculation unit 36 that performs various calculations for calculating the shape or acoustic characteristics of a target 3, based on an electric signal S41 received from the transmission and reception unit 31. The control unit 35 includes a processor (for example, central processing unit (CPU) or a graphics processing unit (GPU)) and a memory in which a program is previously stored. The processor reads and executes the program, and thus realizes the function of the control unit 35. In a case where the control unit 35 is realized by hardware, circuit design may be performed by using a custom IC such as an application specific integrated circuit (ASIC) or a programmable IC such as a field-programmable gate array (FPGA), so as to realize an operation of the control unit 35.

The control unit 35 may perform different controls, for example, control signals S51 and S52, on the transmission and reception units 31. For example, the control unit 35 controls the transmission and reception unit 31 to which the control signal S51 is input, to perform a transmission operation, and controls the transmission and reception unit 31 to which the control signal S52 is input, to perform a reception operation.

The transmission unit 32 is configured by, for example, an amplifier. The transmission unit 32 amplifies an electric signal 51 input from the control unit 35 to have a desired amplitude, and then outputs the amplified signal to the transducer 4a. Acoustic pressure and a transmission timing of an ultrasonic signal S21 transmitted from the transducer 4a are changed in accordance with a transmission timing and a voltage of an electric signal S11 applied to the transducer 4a. Therefore, acoustic pressure and a transmission timing of the ultrasonic signal S21 is controlled in accordance with the amplitude and the transmission timing of the electric signal S1. Alternatively, a gain of the amplifier constituting the transmission unit 32 and a signal response time may be controlled in accordance with the control signal S51, and the voltage and the transmission timing of the electric signal S11 may be controlled.

The reception unit 33 is configured by, for example, a low noise amplifier, a filter, a variable gain amplifier, an analog-digital converter, and the like. An electric signal S31 which is input from the transducer 4a via the transmission and reception switch 34 is amplified by the low noise amplifier. Noise other than a desired frequency band is reduced by the filter, and the resultant is amplified to have an appropriate amplitude by the variable gain amplifier. Then, the resultant signal is converted into a digital signal by the analog-digital converter. An electric signal S41 obtained by conversion into a digital signal is input to the control unit 35. Settings of a circuit constituting the reception unit 33 are controlled in accordance with a control signal S52. An amplitude value (quantized binary number) and a reception timing of the electric signal S41 are changed in accordance with the acoustic pressure and a reception timing of the ultrasonic signal S21. A relationship between the amplitude value and the reception timing of the electric signal S41 and the acoustic pressure and the reception timing of the ultrasonic signal S21 are changed in accordance with the settings of the circuit constituting the reception unit 33. Thus, the relationship thereof may be controlled by the control signal S52 so as to obtain a desired relationship.

The transmission and reception switch 34 cuts off a connection between the reception unit 33 and the transducer 4a during a transmission operation, and has a short circuit between the reception unit 33 and the transducer 4a during a reception operation. Generally, the transmission unit 32 is configured by a high breakdown-voltage transistor in order to output a transmission signal having a high voltage. However, the reception unit 33 is configured by a low breakdown-voltage transistor because amplifying a reception signal having a low voltage. The transmission and reception switch 34 cuts off the connection between the reception unit 33 and the transducer 4a during the transmission operation, such that a high-voltage transmission signal output from the transmission unit 32 is applied to the reception unit 33 configured by a low breakdown-voltage transistor, and thus the reception unit 33 is broken down.

The control unit 35 calculates a transmission and reception result of the ultrasonic signal S21 and calculates the shape or acoustic characteristics of the target 3, with controlling transmission and reception of the ultrasonic signal S21. An ultrasonic signal S21 which has been transmitted from a certain transmission and reception unit 31 via the transducer 4a propagates in the ultrasonic propagation material 6, the water tank 1, the ultrasonic propagation material 2, and the target 3 with being scattered. The ultrasonic signal S21 which has propagated in the above members is received via the same or a different transducer 4a. The shape of the target 3 is calculated by delaying and adding the reception result. A physical property value (sound speed and/or attenuation rate (attenuation amount)) which reflects acoustic characteristics of the target 3 is calculated by using an ultrasonic tomography method.

The control unit 35 stores data indicating the shape of the target 3 and the sound speed and/or the attenuation rate which have been calculated, in the storage unit 38. Various settings such as setting of the transmission unit 32 and setting of the reception unit 33, which are measurement parameters are also stored in the storage unit 38. The control unit 35 reads the various settings from the storage unit 38 and controls the transmission and reception units 31. Further, the control unit 35 performs output of a control command and acquirement of information regarding each of a state of the ultrasonic propagation material 6 and a state of the ultrasonic propagation material 2, for the driving device 14 that moves the measurement unit 49 up and down, the material circulation unit 20 that manages the state of the ultrasonic propagation material 6 in the containment vessel 5, and the material circulation unit 27 that manages the state of the ultrasonic propagation material 2 in the water tank 1.

An operator of the ultrasonic imaging apparatus 52 performs an input and the like of a command via the interface 37. The operator confirms a measurement result of the target 3 by the display unit 39 or confirms various states (setting state, operation state, and the like) of the ultrasonic imaging apparatus 52. The operator may perform communication of information with another device via the interface 37.

Next, an operation of the ultrasonic imaging apparatus 52, particularly, an operation of the control unit 35 will be specifically described with reference to FIGS. 4 to 7. FIGS. 4A and 4B are sequence diagrams illustrating an operation example of the ultrasonic imaging apparatus.

Firstly, if a start instruction is received from the operator (S100), the interface 37 transmits the start instruction to the electronic control device 28 (S101). The electronic control device 28 which has received the start instruction starts an operation as measurement preparation processing. The electronic control device 28 transmits a material unit control instruction to each of the material circulation unit 20 and the material circulation unit 27. The material unit control instruction is a preparation instruction for the ultrasonic propagation material and is provided for controlling filling with the ultrasonic propagation materials 6 and 2, and states (such as the temperature, filling conditions and the like) of the ultrasonic propagation materials 6 and 2 (S102-1 and S102-2). The material circulation unit 20 and the material circulation unit 27 which have received the material unit control instruction fill the water tank 1 and the containment vessel 5 with the ultrasonic propagation materials 6 and 2, respectively. The material circulation unit 20 and the material circulation unit 27 respectively control (manage) the ultrasonic propagation materials 6 and 2 under control of the control unit 35, such that the ultrasonic propagation materials 6 and 2 are in a state suitable for imaging (S111 and S112).

The material unit control instruction includes, for example, a liquid quantity control instruction, a temperature control instruction, and the degassing instruction. The liquid quantity control instruction is an instruction that the control unit 35 respectively instructs the material circulation units 27 and 20 to control the liquid quantities of the ultrasonic propagation materials 2 and 6. The temperature control instruction is an instruction that the control unit 35 respectively instructs the material circulation units 27 and 20 to control the temperature of the ultrasonic propagation materials 2 and 6. The degassing instruction is an instruction that the control unit 35 respectively instructs the material circulation units 27 and 20 to degas oxygen dissolved in the ultrasonic propagation materials 2 and 6.

If the control of filling and the states of the ultrasonic propagation materials 6 and 2 is completed, each of the material circulation unit 20 and the material circulation unit 27 transmits a preparation completion report (S201 and S202). The preparation completion report is used for reporting completion of preparation of the ultrasonic propagation material to the electronic control device 28. Then, the material circulation unit 20 and the material circulation unit 27 continue the control to cause the ultrasonic propagation materials 6 and 2 to respectively maintain the appropriate state, until the ultrasonic imaging apparatus 52 is suspended.

The electronic control device 28 which has received the completion report from both the material circulation unit 20 and the material circulation unit 27 transmits a measurement-unit moving instruction to move the measurement unit 49 to a measurement position, to the driving device 14 (S203). The driving device 14 which has received the measurement-unit moving instruction moves the measurement unit 49 to the measurement position (S113). In a case of the first measurement-unit moving instruction, a moving destination of the measurement unit 49 is a measurement initial position. If the process of S113 is completed, the driving device 14 transmits a moving completion report for reporting completion of moving the measurement unit 49, to the electronic control device 28 (S204).

The electronic control device 28 which has received the moving completion report from the driving device 14 transmits a message indicating that measurement preparation is completed, and an installation instruction to promote installation of a target 3, to the display unit 39 (S103). Then, the display unit 39 displays the message and the installation instruction (S205).

Then, if the target 3 is installed in the water tank 1, the interface 37 receives a measurement start instruction from the operator (S104), and outputs the measurement start instruction to the electronic control device 28 (S206). The electronic control device 28 which has received the measurement start instruction performs control of performing measurement, as measurement execution processing (S105). The electronic control device 28 performs transmission and reception settings for each of the transmission and reception units 31. The electronic control device 28 transmits a transmission signal which is an electric signal for transmitting an ultrasonic wave to the transducer array 4, via each of the transmission and reception units 31 (S207). The transducer array 4 converts the electric signal into an ultrasonic signal, and transmits the ultrasonic signal into the transducer array 4. The transducer array 4 receives an ultrasonic signal from the transducer array 4 (S114).

The transducer array 4 converts the received ultrasonic signal into a reception signal which is an electric signal, and transmits the reception signal to the electronic control device 28 via each of the transmission and reception units 31 (S208).

The electronic control device 28 stores the received reception signal in the storage unit 38. If transmission and reception of the ultrasonic signal in predetermined transmission and reception settings are completed, the electronic control device 28 transmits a measurement-unit moving instruction of moving the measurement unit 49 to the next measurement position, to the driving device 14 (S115). The driving device 14 which has received the measurement-unit moving instruction moves the measurement unit 49 to the next measurement position (S209). If the process of S209 is completed, the driving device 14 transmits a moving completion report to the electronic control device 28 (S210).

The electronic control device 28 which has received the moving completion report transmits a transmission signal to the transducer array 4 via each of the transmission and reception units 31 (S207-1). The electronic control device 28 transmits and receives an ultrasonic wave to the next measurement position at which the measurement unit 49 has moved in S209.

The ultrasonic imaging apparatus 52 repeats transmission and reception (S207, S208, and S114) of an ultrasonic wave and moving (S115, S209, and S210) of the measurement unit 49 until measurement unit 49 reaches the final measurement position. When the measurement unit 49 moves after the measurement unit 49 reaches the final measurement position and performs transmission and reception of an ultrasonic wave, the electronic control device 28 moves the measurement unit 49 to the measurement initial position. Then, the electronic control device 28 transmits a message indicating that the measurement is completed, and a measurement result based on the reception signal stored in the storage unit 38 to the display unit 39 (S106). The display unit 39 displays the message and the measurement result (S211). The electronic control device 28 stores a measurement result in the storage unit 38, and then completes the measurement execution processing.

The operator confirms measurement completion and the measurement result by the display unit 39. Then, the interface 37 receives a washing start instruction by the operator (S107), and outputs the washing start instruction to the electronic control device 28 (S212). The electronic control device 28 which has received the washing start instruction controls the material circulation unit 27 to wash the inside of the water tank 1 (S108). The electronic control device 28 transmits a washing instruction to the material circulation unit 27 (S213). The material circulation unit 27 which has received the washing instruction washes the water tank 1 (S214). Specifically, the material circulation unit 27 drains the ultrasonic propagation material 2 and rinses the inside of the water tank 1 with a new ultrasonic propagation material 2. The material circulation unit 27 cause the ultrasonic propagation material 2 to pass through a filter configured to remove dirt, bacteria, and the like, and thus makes the ultrasonic propagation material 2 be clean. If necessary, the material circulation unit 27 injects a washing liquid into the water tank 1, and thus performs washing. If washing of the water tank 1 is completed, the material circulation unit 27 transmits a washing completion report for reporting completion of washing of the water tank 1 to the electronic control device 28 (S215). The electronic control device 28 which has received the washing completion report transmits a message indicating that washing is completed, and an inquiry of whether or not the ultrasonic imaging apparatus 52 is suspended, to the display unit 39 (S216). The display unit 39 displays the message and the inquiry (S217).

The operator confirms a display of the display unit 39. In a case where the operator suspends the ultrasonic imaging apparatus 52, the interface 37 receives a suspension instruction by the operator (S109). Then, the interface 37 outputs the suspension instruction to the electronic control device 28 (S218). In a case where the ultrasonic imaging apparatus 52 is not to be suspended and measurement of another target 3 continues, the interface 37 receives an execution instruction for instructing execution of the process of S102, by the operator. Then, the interface 37 outputs the execution instruction to the electronic control device 28.

The electronic control device 28 which has received the suspension instruction controls the material circulation unit 20 and the material circulation unit 27 to drain the ultrasonic propagation material 2 in the water tank 1 and the ultrasonic propagation material 6 in the containment vessel 5, as operation suspension processing (S110). The electronic control device 28 transmits a drainage instruction for instructing drainage of the ultrasonic propagation materials 6 and 2, to each of the material circulation unit 20 and the material circulation unit 27 (S219-1 and S219-2).

The material circulation unit 20 and the material circulation unit 27 which have received the drainage instruction perform drainage of the ultrasonic propagation materials 6 and 2, respectively (S220 and S221). If the drainage is completed, each of the material circulation unit 20 and the material circulation unit 27 transmits a drainage completion report to the electronic control device 28 (S222 and S223). The electronic control device 28 which has received the drainage completion report transmits a message indicating an operation of the ultrasonic imaging apparatus 52 is suspended, to the display unit 39 (S224). The display unit 39 displays the message (S225), and the processing is ended.

FIG. 5 is a flowchart illustrating an example of an overall flow of an operation of the control unit. The control unit 35 determines whether or not the start instruction is provided, based on a determination of whether the start instruction is received from the interface 37 (S301). The control unit 35 may determine that the start instruction is provided, by applying power to the ultrasonic imaging apparatus 52. In a case where the start instruction is not provided (No in S301), the control unit 35 determines whether or not the start instruction is provided, again. In a case where the start instruction is provided (Yes in S301), the control unit 35 performs measurement preparation processing (S302 and S303).

The control unit 35 performs material control processing in which the material circulation units 27 and 20 are filled with the ultrasonic propagation materials 2 and 6 and the states (such as the temperature, filling conditions and the like) of the ultrasonic propagation materials 2 and 6 are controlled (S302). Details of S302 will be described later with reference to FIG. 6. The control unit 35 transmits a measurement-unit moving instruction to the driving device 14 and moves the measurement unit 49 to the measurement initial position (S113). The control unit 35 determines whether or not a moving completion report is provided, based on a determination of whether the moving completion report is received from the driving device 14 (S403). In a case where the moving completion report is not provided (No in S403), the control unit 35 determines whether or not the moving completion report is provided, again. In a case where the moving completion report is provided (Yes in S403), the control unit 35 transmits an installation instruction of the target 3 to the display unit 39 (S303).

Then, the control unit 35 determines whether or not a measurement start instruction is provided, based on a determination of whether the measurement start instruction is received from the interface 37 (S304). In a case where the measurement start instruction is not provided (No in S304), the control unit 35 determines whether or not the measurement start instruction is provided, again. In a case where the measurement start instruction is provided (Yes in S304), the control unit 35 performs measurement execution processing (S305). Details of S305 will be described later with reference to FIG. 7.

The control unit 35 determines whether or not the measurement is ended, based on a determination of whether a remeasurement instruction is received from the interface 37 (S306). In a case where the remeasurement instruction is provided (No in S304), the control unit 35 performs the process of S304 again. In a case where the remeasurement instruction is not provided (Yes in S304), the control unit 35 determines whether or not a washing start instruction is provided, based on a determination of whether the washing start instruction is received from the interface 37 (S307). In a case where the washing start instruction is not provided (No in S307), the control unit 35 determines whether or not the washing start instruction is provided, again. In a case where the washing start instruction is provided (Yes in S307), the control unit 35 performs washing processing of the inside of the water tank (S308). Specifically, corresponding to the process of S108 in FIG. 4, the control unit 35 transmits a washing instruction to the material circulation unit 27. After washing of the water tank 1 is completed by the material circulation unit 27, the control unit 35 receives a washing completion report from the material circulation unit 27 and transmits a message indicating washing is completed, and an inquiry of whether or not the ultrasonic imaging apparatus 52 is suspended, to the display unit 39.

The control unit 35 determines whether or not a suspension instruction is provided, based on a determination of whether the suspension instruction is received from the interface 37 (S309). In a case where the suspension instruction is not provided (No in S309), the control unit 35 performs the process of S302 again. In a case where the suspension instruction is provided (Yes in S309), the control unit 35 performs drainage processing of the ultrasonic propagation material (S310). Specifically, corresponding to the process of S110 in FIG. 4, the control unit 35 transmits a drainage instruction to the material circulation units 20 and 27. After drainage of the ultrasonic propagation materials 6 and 2 is respectively completed by the material circulation units 20 and 27, the control unit 35 receives a drainage completion report from each of the material circulation units 20 and 27, and transmits a message indicating that an operation of the ultrasonic imaging apparatus 52 is suspended, to the display unit 39.

FIG. 6 is a flowchart illustrating an example of details of the material control processing by the control unit.

Firstly, the control unit 35 transmits filling instructions as material unit control instructions to the material circulation units 27 and 20, respectively (S401). The filling instructions are an instruction to fill the water tank 1 with the ultrasonic propagation material 2 and an instruction to fill the containment vessel 5 with the ultrasonic propagation material 6.

Then, the control unit 35 detects a liquid level of the ultrasonic propagation material 2 in the water tank 1 and a liquid level of the ultrasonic propagation material 6 in the containment vessel 5 (S116). That is, the control unit 35 detects whether or not there are the liquid levels of the ultrasonic propagation materials 2 and 6.

Regarding the ultrasonic propagation material 2, the control unit 35 detects the liquid level of the ultrasonic propagation material 2 by using a liquid level sensor which is installed in the vicinity of the opening portion of the water tank 1 although not illustrated. Regarding the ultrasonic propagation material 6, the control unit 35 detects the liquid level of the ultrasonic propagation material 6 by using the liquid level sensor 19. The amounts of the ultrasonic propagation materials 2 and 6 which have been respectively injected into the water tank 1 and the containment vessel 5 may be recognized by using a flowmeter and the recognized amount may be compared to the amount corresponding to a desired liquid level.

Then, the control unit 35 determines whether or not a detection result of each of the liquid levels corresponds to a predetermined liquid level (S117). Ina case where the liquid level is not detected in S116, this case means that the liquid level of the material does not reach the predetermined liquid level. In this case (No in S117), the control unit 35 controls the liquid quantity (S118). Specifically, the control unit 35 transmits a liquid quantity control instruction as a material unit control instruction to the material circulation unit 27 or the material circulation unit 20, and continues injection of the ultrasonic propagation material 2 or 6 (S118). In a case where detection of the liquid level of the ultrasonic propagation material 6 is not possible even though a predetermined time waits, there is a probability of the liquid-tight structure 9 having a problem or the like. Thus, the control unit 35 may notify the operator of the message indicating that there is a probability of the liquid-tight structure 9 having a problem, by using an alert or the like.

In a case where the liquid level is detected in S116, this case means that the liquid level reaches a predetermined liquid level. In this case (Yes in S117), then, the control unit 35 detects the temperature of each of the ultrasonic propagation materials 2 and 6 (S119). A temperature sensor (not illustrated) or the like is provided on the bottom surface of the water tank 1, and thus the control unit 35 detects the temperature of the ultrasonic propagation material 2 in the water tank 1. The control unit 35 detects the temperature of the ultrasonic propagation material 6 in the containment vessel 5 by the temperature sensor 18.

Then, the control unit 35 determines whether or not the detected temperature is in a predetermined temperature range (S120). In a case where the detected temperature is not in the predetermined temperature range (No in S120), temperature control is performed (S121) . Specifically, the control unit 35 transmits a temperature control instruction as the material unit control instruction to the material circulation unit 20, and controls the temperature control device 23 to heat or cool the ultrasonic propagation material 6. The above descriptions are similarly applied to a case of the ultrasonic propagation material 2. The control unit 35 transmits a temperature control instruction as the material unit control instruction to the material circulation unit 27, and controls a temperature control device (not illustrated) in the material circulation unit 27 to heat or cool the ultrasonic propagation material 2.

For example, in a case where the temperature of the ultrasonic propagation material 2 in the containment vessel 5 is held to be 37 degrees which is substantially equal to the body temperature of a person, the temperature is controlled as follows. Heat of the ultrasonic propagation material 2 in the containment vessel 5 propagates to the containment vessel 5, the water tank 1, the hose 7, and the like and is diffused to the peripherals. In a case where the measurement target is a person, since the ultrasonic imaging apparatus 52 is installed in a space having a temperature (for example, pleasant temperature of about 25 degrees) which is lower than 37 degrees, the temperature of the ultrasonic propagation material 2 in the containment vessel 5 is decreased over time.

The temperature control device 23 continuously heats the ultrasonic propagation material 2 in the material reservoir 21 to be about 37 degrees, such that the temperature of the ultrasonic propagation material 2, which is detected by the temperature sensor 18 is decreased from 37 degrees by a predetermined value or greater. If the temperature of the ultrasonic propagation material 2 is increased from 37 degrees by a predetermined value or greater, due to a certain cause, the ultrasonic propagation material 2 in the material reservoir 21 is cooled to be about 37 degrees by the temperature control device 23. The above descriptions are similarly applied to a case of the ultrasonic propagation material 2 in the water tank 1. The ultrasonic propagation material 2 in the material circulation unit 27 is continuously heated to be about 37 degrees by the temperature control device which is provided in the material circulation unit 27 although not illustrated, so as to compensate for heat which propagates to the opening portion at the upper portion of the water tank 1 or the wall thereof, the hose 8, the target 3, and the like.

In a case where the detected temperature is in the predetermined temperature range (Yes in S120), then, the control unit 35 detects dissolved oxygen concentration in the ultrasonic propagation material for each of the ultrasonic propagation materials 2 and 6 (S122). The control unit 35 detects the dissolved oxygen concentration for each of the ultrasonic motorized members 2 and 6 by using a dissolved oxygen concentration meter (not illustrated) disposed at a position which is similar to that of the temperature sensor. Then, the control unit 35 determines whether or not the detected dissolved oxygen concentration is equal to or smaller than predetermined dissolved oxygen concentration (S123). In a case where the detected dissolved oxygen concentration is higher than the predetermined dissolved oxygen concentration (No in S123), degassing continues (S124).

Specifically, the control unit 35 transmits a degassing instruction as the material unit control instruction to the material circulation unit 20. The control unit 35 performs degassing of the ultrasonic propagation material 6 by causing the ultrasonic propagation material 6 to pass through the degassing device 24. The above descriptions are similarly applied to a case of the ultrasonic propagation material 2. The control unit 35 transmits the degassing instruction as the material unit control instruction to the material circulation unit 27. The control unit 35 performs degassing of the ultrasonic propagation material 2 by causing the ultrasonic propagation material 2 to pass through a degassing device (not illustrated) in the material circulation unit 27.

In a case where the detected dissolved oxygen concentration is equal to or smaller than the predetermined dissolved oxygen concentration (Yes in S123), since each of the ultrasonic propagation materials 2 and 6 is managed to be in a predetermined state, measurement can start. Therefore, the control unit 35 determines whether or not a preparation completion report is provided, based on a determination of whether the preparation completion report is received from each of the material circulation units 20 and 27 (S402). In a case where the preparation completion report is not provided (No in S402), the control unit 35 determines whether or not the preparation completion report is provided, again. In a case where the preparation completion report is provided (Yes in S402), the control unit 35 ends the material control processing and causes the process to proceed to S113.

FIG. 6 illustrates a flow of sequentially performing liquid level position detection (S116), temperature detection (S119), and dissolved oxygen concentration detection (S122). However, in practice, the processes may be performed in parallel with each other. Even if the detected value is a predetermined value once, detection and control may be continuously performed until the ultrasonic imaging apparatus 52 is suspended. The measurement unit 49 is moved to the measurement initial position for the first time, and then the liquid level position, the temperature, and the dissolved oxygen concentration may be detected and controlled.

FIG. 7A is a flowchart illustrating an example of details of the measurement execution processing by the control unit. Firstly, the control unit 35 reads the collected measurement parameters which are transmission and reception settings such as the setting of the transmission and reception unit 31 and position coordinates of the transducer 4a, from the storage unit 38 (S125). Then, the control unit 35 performs transmission setting regarding transmission of an ultrasonic wave, in the transmission and reception unit 31 which is connected to the transducer (transmission transducer) 4a that transmits the ultrasonic wave, and performs reception setting regarding reception of an ultrasonic wave, in the transmission and reception unit 31 which is connected to the transducer (reception transducer) 4a that receives the ultrasonic wave (S126). The settings are performed based on the measurement parameters which have been read. The control unit 35 transmits a transmission signal to the transmission transducer 4a via the transmission and reception unit 31 in which the transmission setting is performed in S126, and cause an ultrasonic signal to be transmitted from this transmission transducer 4a (S127). The control unit 35 causes the reception transducer 4a connected to the transmission and reception unit 31 in which the reception setting is performed in S126, to receive an ultrasonic signal. A reception signal is received from this reception transducer 4a (S128). The control unit 35 stores the received reception signal as reception signal data, in the storage unit 38 (S129).

The transmission and reception unit 31 in which the transmission setting is performed in S126 and the transmission transducer 4a can transmit an ultrasonic signal, and then can receive an ultrasonic signal based on the reception setting.

Then, the control unit 35 determines whether all sets of transmission and reception settings which have been collectively read are performed, that is, whether or not the current set of the transmission and reception settings is a final set of the transmission and reception settings (S130). In a case where a set of the transmission and reception settings remains (No in S130), the control unit 35 changes the current set of the transmission and reception settings to the next set of the transmission and reception settings (S131). The process returns to S126. The control unit 35 performs the process subsequent to the process of S126 based on the changed set of the transmission and reception settings, and thus performs transmission and reception of an ultrasonic signal.

In a case where all sets of the transmission and reception settings are performed (Yes in S130), the calculation unit 36 of the control unit 35 calculates the shape of the target 3 and acoustic characteristic distribution based on the reception signal data in each set of the transmission and reception settings, which has been recorded in S129 (S132). At this time, the control unit 35 may store a calculation result in the storage unit 38. The calculation of the shape and the acoustic characteristic distribution of the target 3 means, for example, that the shape is calculated by delaying and adding the reception signal data of the ultrasonic signal, and means that the acoustic characteristic distribution is calculated by using a tomography method.

After the process of S132, the control unit 35 determines whether or not the measurement unit 49 reaches predetermined final measurement position (S133). In a case where the measurement unit 49 does not reach the final measurement position (No in S133), the control unit 35 initializes the set of the transmission and reception settings and changes the position of the measurement unit 49 (S134). Specifically, the control unit 35 brings the set of the transmission and reception settings back to the first settings. The control unit 35 transmits a measurement-unit moving instruction to the driving device 14. The control unit 35 moves the measurement unit 49 to the next measurement position by changing the position of the measurement unit 49 by a predetermined step width. The process returns to S126, and the processes subsequent to the process of S126 are performed. The control unit 35 repeats transmission and reception of an ultrasonic signal by using all sets of the transmission and reception settings in the next measurement position.

In a case where the measurement unit 49 reaches the final measurement position (Yes in S133), this means that measurement of the target 3 is completed. Thus, the control unit 35 prepares the next measurement in a manner that the control unit 35 transmits a measurement-unit moving instruction to the driving device 14 and moves the measurement unit 49 to the measurement initial position (S135). The control unit 35 transmits all measurement results to the display unit 39 and causes all of the measurement results to be displayed in the display unit 39. The control unit 35 stores data of the final measurement result in the storage unit 38 (S136), and ends the measurement execution processing. Then, the control unit 35 causes the process to proceed to S306. As the final measurement result, the three-dimensional shape of the target 3, three-dimensional acoustic characteristic distribution, and the like are provided.

FIG. 7B is a flowchart illustrating a modification example of the details of the measurement execution processing by the control unit. FIG. 7A illustrates an operation example in which transmission and reception of an ultrasonic wave and moving of the measurement unit 49 are alternately performed. However, FIG. 7B illustrates an operation example in which transmission and reception of an ultrasonic wave and moving of the measurement unit 49 are simultaneously performed. Descriptions will be made focusing on a difference from FIG. 7A. The same part is denoted by the same reference sign in FIG. 7A and descriptions thereof will not be repeated.

After the process of S125, the control unit 35 transmits a measurement-unit moving instruction to the driving device 14 and starts moving of the measurement unit 49 (S137).

The moving of the measurement unit 49 is performed sufficiently slow and continuously. Thus, similar to helical scanning which is performed by an X-ray CT apparatus, it is possible to obtain a spiral ultrasonic transmission and reception result for the target 3. Since transmission and reception of an ultrasonic wave and moving of the measurement unit 49 are simultaneously performed, it is possible to reduce a time required for measurement.

After the process of S129, the control unit 35 determines whether or not the measurement unit 49 reaches the final measurement position (S133). In a case where the measurement unit 49 does not reach the final measurement position (No in S133), the control unit 35 changes the current set of the transmission and reception settings to the next set of the transmission and reception settings (S131) and causes the process to return to S126. In a case where the measurement unit 49 reaches the final measurement position (Yes in S133), t e calculation unit 36 of the control unit 35 performs the process of S132 so as to calculate the shape of the target 3 and acoustic characteristic distribution. After the process of S132, the control unit 35 performs the processes of S135 and S136, and ends the measurement execution processing.

Then, calculation of the shape of the target 3 and acoustic characteristics in consideration of an ultrasonic wave propagation path of an ultrasonic signal will be described with reference to FIGS. 8 to 13.

FIG. 8 is a diagram illustrating an ultrasonic wave propagation path of an ultrasonic signal. FIG. 8A is a diagram illustrating an ultrasonic wave propagation path in a case where the transducer array is installed in the water tank. FIG. 8B is a diagram illustrating an ultrasonic wave propagation path through the water tank, the ultrasonic propagation material in the water tank, and the ultrasonic propagation material in the containment vessel. FIG. 8C is a diagram illustrating directivity of a signal delayed time of an ultrasonic signal.

In the related art, a signal delayed time from transmission until reception by using a pair of transducers 4a is measured. A propagation distance is calculated from a propagation speed of an ultrasonic wave, and thus position coordinates of the transducers 4a are estimated and corrected. If the water tank 1 into which the target 3 is inserted and the containment vessel 5 which stores the transducer array 4 are separated from each other, the ultrasonic propagation material 2 in the water tank 1, the wall of the water tank 1, and the ultrasonic propagation material 6 in the containment vessel 5 are provided during a period from transmission until reception by using a pair of transducers 4a. Thus, in the related art, it is not possible to correct the position coordinates of the transducer 4a with high accuracy. As a result, it is not possible to calculate the shape of the target 3 and the acoustic characteristic distribution with high accuracy. This is because an ultrasonic wave is refracted by a difference of an ultrasonic wave propagation speed or density thereof or a relationship between a propagation time and a propagation distance varies depending on a transmission angle of an ultrasonic wave.

In the configuration in FIG. 8A, an ultrasonic signal S21 transmitted from the transducer 4a installed in the water tank 1 straightly propagates in the ultrasonic propagation material 2 which has a constant sound speed and uniform density. Therefore, it can be considered that the position of the transducer 4a corresponds to the position of a wave source 53.

On the contrary, in the configuration in FIG. 8B which is the configuration of this example, an ultrasonic signal S21 transmitted from the transducer 4a installed in the containment vessel 5 propagates in media having three types of sound speeds and density, that is, the ultrasonic propagation material 6, the wall of the water tank 1, and the ultrasonic propagation material 2.

The ultrasonic propagation material 2 and the ultrasonic propagation material 6 can have the same composition and the same condition. However, the water tank 1 has a sound speed and density which are different from those of the ultrasonic propagation materials 2 and 6. Accordingly, an ultrasonic signal S21 transmitted from the transducer 4a is refracted and propagates at an interface between the ultrasonic propagation material 6 and the wall of the water tank 1 and at an interface between the wail of the water tank 1 and the ultrasonic propagation material 2. The water tank 1 has a sound speed which is different from those of the ultrasonic propagation materials 2 and 6. Thus, a propagation distance of an ultrasonic signal S21a is different from propagation distances of ultrasonic signals S21b and S21c at the same time point. Therefore, it is necessary that it is considered that the position of the transducer 4a corresponds to the position of a virtual wave source 54 not the position of the wave source 53.

Further, a distance when an ultrasonic signal S21 passes through the wall of the water tank 1 varies depending on a propagation direction of the ultrasonic signal S21. Thus, the ultrasonic signal S21 has directivity of a signal delayed time as illustrated in FIG. 8C.

FIG. 9 is a diagram illustrating a signal propagation path of the ultrasonic imaging apparatus.

The calculation unit 36 detects a signal delayed time T1 which is a difference between an output timing of an electric signal S1 by the control unit 35 and an input timing of an electric signal S41 from the reception unit 33.

The electric signal S1 is transmitted as the ultrasonic signal S21 from the transducer 4a after a signal response time T2 elapses after being input to the transmission unit 32. The signal response time T2 is determined in accordance with electric characteristics of the transmission unit 32 and electric-ultrasonic conversion characteristics of the transducer 4a. The ultrasonic signal S21 transmitted from a transmission side transducer 4a is received in a reception side transducer 4a after a time elapses. The time satisfies a signal propagation time T3 taken to propagate in the ultrasonic propagation materials 2 and 6 and the wall of the water tank 1 (propagation time on the assumption of the virtual wave source 54 and the ultrasonic propagation materials 2 and 6) and directivity (signal delayed time based on the propagation direction of the ultrasonic signal S21) T5 of the signal delayed time illustrated in FIG. 8C.

The directivity T5 of the signal delayed time also similarly occurs in the reception side transducer 4a in addition to the transmission side transducer 4a. The ultrasonic signal S21 received in the reception side transducer 4a is output as an electric signal S41 from the reception unit 33 to the calculation unit 36 after a signal response time T4 elapses. The signal response time T4 is determined in accordance with ultrasonic-electric conversion characteristics of the transducer 4a and electric characteristics of the reception unit 33.

From the above descriptions, in order to accurately measure a signal propagation time T3 and to calculate the shape of the target 3 and acoustic characteristics with high accuracy, it is necessary that a correction parameter is required. The correction parameter relates to position coordinates of the virtual transducer 4a, the directivity T5 of the signal delayed time, the signal response time T2 of the transmission unit 32, and the signal response time T4 of the reception unit 33. The typical value of the correction parameter is obtained by typical electric characteristics, typical dimensions, or the like of the transmission and reception unit 31, the transducer array 4 and the water tank 1. However, manufacturing variations occur in the above factors. Thus, in order to calculate the shape of the target 3 and the acoustic characteristics with higher accuracy, it is necessary that a correction parameter calculation operation of the measurement unit 49 illustrated in FIG. 10 is performed in a state where the target 3 is not installed.

FIG. 10 is a flowchart illustrating an example of the correction parameter calculation operation of the measurement unit, which is performed by the control unit and in which the wall of the water tank and the ultrasonic propagation material in the containment vessel are provided. Parts which are the same as those in FIGS. 5 to 7 are denoted by the same reference signs and descriptions thereof will not be repeated.

In S302, a preparation completion report is received from each of the material circulation units 20 and 27 (Yes in S402), and preparation of the ultrasonic propagation material 2 in the water tank 1 and the ultrasonic propagation material 6 in the containment vessel 5 is finished. Then, the control unit 35 transmits a measurement-unit moving instruction for correction, which is used for movement to an correction initial position, to the driving device 14, and moves the measurement unit 49 to the correction initial position (S148).

Then, the control unit 35 reads a correction initial parameter from the storage unit 38 (S149). The correction initial parameter is set to be a typical value which relates to the position coordinates of the virtual transducer 4a, the directivity T5 of the signal delayed time, the signal response time T2 of the transmission unit 32, and the signal response time T4 of the reception unit 33.

Then, the control unit 35 sequentially transmits and receives an ultrasonic signal S21 for all preset pairs of transducers 4a, and acquires reception signal data to be used when the signal delayed time T1 is calculated (S126 to S131). The calculation unit 36 in the control unit 35 performs correction parameter calculation processing based on the acquired reception signal data by using a least-square method or the like. In the correction parameter calculation processing, the signal delayed time T1 and the correction parameter which relates to the position coordinates of the virtual transducer 4a, the directivity T5 of the signal delayed time, the signal response time T2 of the transmission unit 32, and the signal response time T4 of the reception unit 33 are calculated (S150). Details of S150 will be described later with reference to FIG. 11.

After the process of S150, the control unit 35 determines whether or not the measurement unit 49 reaches the predetermined final measurement position (S133). In a case where the measurement unit 49 does not reach the final measurement position (No in S133), the control unit 35 initializes the set of the transmission and reception settings and changes the position of the measurement unit 49 (S134). The control unit 35 performs a series of operations from S126 to S150 in all measurement positions of the measurement unit 49, for all sets of transmission and reception settings.

If the calculation of the correction parameter is completed for all measurement positions of the measurement unit 49 (Yes in S133), the control unit 35 transmits a measurement-unit moving instruction to the driving device 14 and thus moves the measurement unit 49 to the measurement initial position (S135). The control unit 35 transmits the final correction result to the display unit 39, causes the final correction result to be displayed in the display unit 39, and stores data of the final correction result in the storage unit 38 (S151). The control unit 35 is in a state of waiting for reception of a measurement start instruction from the interface 37. If the control unit 35 receives the measurement start instruction, the control unit 35 performs, for example, the processes subsequent to the process of S304 (S180).

The correction parameter other than the directivity T5 of the signal delayed time is constant regardless of the position of the measurement unit 49. Thus, in S151, the position coordinates of the virtual transducer 4a and the signal response times T2 and T4 which are most reliable may be calculated based on correction results at all positions of the measurement unit 49 by using the least-square method in a manner similar to that in S150. Then, the position coordinates and the signal response times T2 and T4 which have been calculated may be set as the final correction parameter along with the directivity T5 of the signal delayed time.

FIG. 11 is a flowchart illustrating an example of details of the correction parameter calculation processing by the control unit . Firstly, the calculation unit 36 in the control unit 35 calculates an initial signal delayed time T1a by using the correction initial parameter which has been read in S149 (S152). Then, the calculation unit 36 calculates the signal delayed time T1 by using reception signal data which is an ultrasonic wave transmission and reception result for correction which has been acquired in S126 to S131 (S153). The calculation unit 36 calculates a difference between the initial signal delayed time T1a and the calculated signal delayed time T1 (S154). Then, the calculation unit 36 determines whether or not the square sum of the signal delayed time difference is greater than a predetermined value (S155).

In a case where the square sum is smaller than the predetermined value (No in S155), the correction initial parameter has accuracy which is sufficient as the correction parameter. Thus, the calculation unit 36 employs the correction initial parameter as the correction parameter, and ends calculation of the correction parameter. Then, the process proceeds to S133. In a case where the square sum is greater than the predetermined value (Yes in S155), the correction initial parameter does not have accuracy which is sufficient as the correction parameter. Thus, the calculation unit 36 causes the process to proceed to steps subsequent to the process of S156. Thus, the calculation unit 36 calculates a more suitable parameter, and updates the calculated parameter as the correction parameter.

Firstly, in S156, the calculation unit 36 obtains an average value of differences between the initial signal delayed time T1a and the calculated signal delayed time T1 for pairs of transducers 4a. The calculation unit 36 calculates the directivity T5 of the signal delayed time as a tendency of the entirety of the transducer array 4, and updates the calculated directivity T5 as the correction Parameter (S156). The calculation unit 36 calculates the signal delayed time T1a by using the updated correction parameter instead of the correction initial parameter, and thus obtains a difference between the signal delayed time T1a and the calculated signal delayed time T1. The calculation unit 36 calculates virtual position coordinates of a specific transducer 4a, which cause the square sum of the difference between the signal delayed time T1 and the signal delayed time T1a calculated by using the updated correction parameter to be smallest. Then, the calculation unit 36 updates the correction parameter (S157). Then, the calculation unit 36 calculates the signal delayed time T1a again by using the updated correction parameter. The calculation unit 36 calculates virtual signal response times T2 and T4 for the same transducer 4a, which cause the square sum of the difference between the signal delayed time T1a and the signal delayed time T1 to be smallest. Then, the calculation unit 36 updates the correction parameter (S158).

The calculation unit 36 performs calculations in S157 and S158 and update of the correction parameter for all transducers 4a. The calculation unit 36 determines whether or not the calculation is ended (S159). In a case where there is a transducer 4a for which the above processes are not performed yet (No in S159), the calculation unit 36 changes a transducer 4a as the target (S160) and causes the process to return to S157. Then, the calculation unit 36 performs the processes of S157 and S158.

In a case where the calculation for all of the transducers is ended (Yes in S159), the calculation unit 36 calculates a difference between the signal delayed time T1a calculated from a calculation result, and the calculated signal delayed time T1 (S161). Similar to S156, the calculation unit 36 calculates the directivity T5 of the signal delayed time as the tendency of the entirety of the transducer array 4, and updates the calculated directivity T5 as the correction parameter (S181).

The calculation unit 36 determines whether or not the calculation in S181 and update of the correction parameter are performed a predetermined number of times (S162). In a case where the above processes are not performed the predetermined number of times (No in S162), the process returns to S155 and the processes subsequent to S155 are performed. In a case where the above processes are performed the predetermined number of times (Yes in S162), the calculation unit 36 ends the calculation of the correction parameter and causes the process to proceed to S133. As described above, even in a case where the transducer array 4 is disposed on the outside of the water tank 1, it is possible to calculate the shape of the target 3 and the acoustic characteristic distribution with higher accuracy by calculating and updating the correction parameter.

FIG. 12 is an example of a flowchart illustrating a modification example of the correction parameter calculation operation of the measurement unit which includes the wall of the water tank and the ultrasonic propagation material in the containment vessel. The correction parameter calculation operation is performed by the control unit. In this flowchart, another operation and determination are added to the operations of S126 to S129 in FIG. 10, and thus it is detected whether or not bubbles are provided in the ultrasonic propagation material 6 in the containment vessel 5 or on the wall surface of the water tank 1. In a case where bubbles are detected, the bubbles are removed and then correction is performed again. Descriptions will be made focusing on a difference from FIG. 10.

After the process of S149, the calculation unit 36 determines whether or not the final reception setting is completed for one transmission setting which has been performed in the previous processes of S126 to S129, that is, whether or not all reception settings are completed for one transmission setting (S163). After all of the reception settings are completed for one transmission setting (Yes in S163), the calculation unit 36 calculates reception signal intensity distribution for the transmission setting (S164). Here, all of the reception settings for one transmission setting means, for example, a case where one specific transducer 4a is set as the transmission side transducer 4a and all the remaining transducers 4a are set as the reception side transducers 4a.

The calculation unit 36 determines whether or not the calculated reception signal intensity distribution is in a range of predetermined distribution (S165). In a case where the calculated reception signal intensity distribution is in the range of predetermined distribution (Yes in S165), the process proceeds to S130 in FIG. 10. In a case where the reception signal intensity distribution is not in the range of the predetermined distribution (No in S165), the calculation unit 36 determines whether this state is repeated a predetermined number of times, that is, the reception signal intensity distribution is not in the range of the predetermined distribution for the predetermined number of times (S166). In a case where it is determined that this state is repeated the predetermined number of times (Yes in S166), the transducer array 4 or the water tank 1 is broken or a situation in which bubbles of which removal is not possible are provided occurs. Thus, the calculation unit 36 displays an alert in the display unit 39 (S168). If the number of times of repeating does not reach the predetermined number of times (No in S166), there is a probability of bubbles being provided on the wall of the water tank 1 or in the ultrasonic propagation material 6. Thus, the control unit 35 moves the measurement unit 49 up or down, and attempts to remove bubbles by causing the wall of the water tank 1 to be rubbed on the hole structure 10 or the liquid-tight structure 9. Then, the control unit 35 brings the measurement unit 49 back to the original position (S167). After such processes are performed, the control unit 35 performs the process of S126 to S129 regarding transmission and reception of an ultrasonic wave, again. Thus, it is possible to avoid an occurrence of a situation in which an erroneous correction parameter is set due to the presence of bubbles. As a result, it is possible to measure the target 3 with higher accuracy.

FIG. 13 illustrates an example of the predetermined distribution in S165 in FIG. 12. FIG. 13A illustrates distribution in which bubbles are not provided on the wall of the water tank 1 or in the ultrasonic propagation material 6, but variation in reception signal intensity is shown by manufacturing variation of the transmission and reception unit 31 or the transducer 4a. The reception signal intensity is provided in a range indicated by a dot line. On the contrary, in a case where bubbles are provided on the wall of the water tank 1 or in the ultrasonic propagation material 6, a state like FIGS. 13B, 13C, and 13D occurs.

FIG. 13B illustrates a case where bubbles are provided at a Position which is very close to the transmission side transducer 4a. The ultrasonic signal S21 transmitted from the transmission side transducer 4a is scattered at almost angles by bubbles, and thus the reception signal intensity is significantly decreased. FIG. 13C illustrates a case where bubbles are provided at a position which is very close to a specific reception side transducer 4a. In this case, the reception signal intensity is significantly decreased only at an angle corresponding to the specific reception side transducer 4a. FIG. 13D illustrates a case where many fine bubbles are provided in the ultrasonic propagation material 6 or the ultrasonic propagation material 2. In this case, variation in reception signal intensity occurs significantly largely depending on the angle. As described above, the distribution of the reception signal intensity is determined, and thus it is possible to estimate whether or not bubbles are provided.

FIG. 14 is a diagram illustrating a modification example of the measurement unit and the material circulation unit configured to circulate the ultrasonic propagation material in the measurement unit. In FIG. 14, the illustration of the position detection sensor 17 is omitted.

FIG. 14A illustrates a configuration in which a liquid level sensor 40 different from the liquid level sensor 19 is installed on the outside of the wall on the upper surface of the containment vessel 5, and a material circulation unit 41 in which a valve 42 is provided at the hose 7 connected to the upper portion of the containment vessel 5 is provided, in comparison to FIG. 2D.

FIG. 14B illustrates a configuration in which the liquid level sensor 40 different from the liquid level sensor 19 is installed on the outside of the wall on the upper surface of the containment vessel 5 and a material circulation unit 43 is provided, in comparison to FIG. 2D. In the material circulation unit 43, a pump 22 and a degassing device 24 are installed on the hose 7 connected to the upper portion of the containment vessel 5 so as to cause the ultrasonic propagation material 6 to flow in the containment vessel 5, a valve 42 is installed on the hose 7 connected to the lower portion of the containment vessel 5 so as to cause the ultrasonic propagation material 6 to flow out to the material reservoir 21.

With the configuration in FIG. 14A or FIG. 14B, the control unit 35 can detect the ultrasonic propagation material 6 which remains on the upper surface of the containment vessel 5, by the liquid level sensor 40. The control unit 35 can control the valve 42 so as to control the amount of the ultrasonic propagation material 6 such that the ultrasonic propagation material 6 does not overflow.

FIG. 15 is a flowchart illustrating an operation example of the control unit in which control is performed so as to cause the ultrasonic propagation material remaining on the upper surface of the containment vessel not to overflow in the configuration illustrated in FIG. 14.

Firstly, the control unit 35 confirms outputs of the liquid level sensors 19 and 40 (S138). The control unit 35 detects whether or not the liquid level of the ultrasonic propagation material 6 is lower than an upper limit liquid level at which the ultrasonic propagation material 6 does not overflow from the upper surface of the containment vessel 5. The detection is performed based on the output of the liquid level sensor 40 (S139). In a case where the liquid level thereof is lower than the upper limit liquid level (Yes in S139), the control unit 35 detects whether or not the liquid level of the ultrasonic propagation material 6 is higher than a lower limit liquid level suitable for measurement, based on the output of the liquid level sensor 19 (S140). In a case where the liquid level thereof is higher than the lower limit liquid level (Yes in S140), the control unit 35 ends this control because the liquid level of the ultrasonic propagation material 6 is in a desired range. The control unit 35 repeats the control in FIG. 15 at a predetermined cycle during the measurement preparation processing and during the measurement execution processing.

In a case where the liquid level thereof is higher than the upper limit liquid level in S139 (No in S139), or in a case where the liquid level thereof is lower than the lower limit liquid level in S140 (No in S140), the control unit 35 stores a period when the state continues, in the storage unit 38. The control unit 35 determines whether or not the state continues during a predetermined period or for a predetermined number of times (S141).

In a case where the state of No in S139 or No in S140 continues during the predetermined period or for the predetermined number of times (Yes in S141), the control unit 35 outputs an alert to the display unit 39 because there is a probability of an occurrence of abnormality in the measurement unit 49, the material circulation units 41 or 43, the hose 7, or the like. For example, a portion of the measurement unit 49 or the hose 7 is broken and thus liquid leakage is caused. Thus, for example, the liquid level of the ultrasonic propagation material 6 is required to be higher than the lower limit liquid level. In a case where the period is shorter than the predetermined period or the number of times is smaller than the number of times of determinations in S141 (No in S141), the control unit 35 transmits a material unit control instruction to the material circulation units 41 and 43. The control unit 35 controls the pump 22 to control the inflow amount of the ultrasonic propagation material 6 into the containment vessel 5 or controls the valve 42 to control the outflow amount of the ultrasonic propagation material 6 from the containment vessel 5. Then, the control unit 35 performs the process of S138 again, and confirms the liquid level of the ultrasonic propagation material 6.

With the configuration in FIGS. 14 and 15, the transducer array 4 can be managed such that the liquid level of the ultrasonic propagation material 6 is maintained to have a position which is appropriately for the position of the transducer array 4. It can be detected whether or not abnormality occurs in the measurement unit 49, the hose 7, the material circulation units 41 and 43, or the like. Thus, it is possible to secure certainty of the measurement result.

FIG. 16 is a diagram illustrating another modification example of the measurement unit and the material circulation unit configured to circulate the ultrasonic propagation material in the measurement unit. In FIG. 16, the illustration of the position detection sensor 17 is omitted. FIG. 16A illustrates a configuration of including a material circulation unit 44 in which a flowmeter 45 is installed on the hose 7 connected to the upper portion of the containment vessel 5 and a flowmeter 45 is installed on the hose 7 connected to the lower portion of the containment vessel 5, in comparison to FIG. 14A. FIG. 16B illustrates a configuration of including a material circulation unit 46 in which a flowmeter 45 is installed on the hose 7 connected to the upper portion of the containment vessel 5 and a flowmeter 45 is installed on the hose 7 connected to the lower portion of the containment vessel 5, in comparison to FIG. 14B. With the configuration in FIG. 16A or FIG. 16B, it is possible to manage the inflow amount and the outflow amount of the ultrasonic propagation material 6 into and from the containment vessel 5. It is possible to fill the containment vessel 5 with the appropriate amount of the ultrasonic propagation material 6 while it is detected whether or not liquid leakage of the ultrasonic propagation material occurs. The flow in FIG. 15 can be also applied to the configuration in FIG. 16.

FIG. 17 is a flowchart illustrating an operation example of the control unit that detects whether or not liquid leakage of the ultrasonic propagation material occurs in the containment vessel or the hose in the configuration illustrated in FIG. 16. The control unit 35 repeats the control in FIG. 17 during the measurement preparation processing and during the measurement execution processing.

Firstly, the control unit 35 confirms outputs of the two flowmeters 45 (S144). Then, the control unit 35 determines whether or not the inflow amount of the ultrasonic propagation material 6 into the containment vessel 5 is greater than the outflow amount of the ultrasonic propagation material 6 from the containment vessel 5 by a predetermined value (S145). In a case where the inflow amount thereof is greater than the outflow amount thereof (Yes in S145), there is a probability of the ultrasonic propagation material 6 being leaked in any of the containment vessel 5 and the hose 7. In this case, the control unit 35 stores a period when this state continues, in the storage unit 38. The control unit 35 determines whether or not the state continues during a predetermined period or for a predetermined number of times (S146). In a case where the state of Yes in S145 continues during the predetermined period or for the predetermined number of times (Yes in S146), the control unit 35 displays an alert in the display unit 39 (S147).

If it is determined to be No (No in S145 and No in S146) in any of S145 and S146, it is considered that no leakage of the ultrasonic propagation material 6 occurs in the hose 7 or the containment vessel 5 or leakage thereof occurs temporarily via the liquid-tight structure 9 with moving the measurement unit 49 up and down. Thus, there is no problem. The ultrasonic propagation material 6 leaked from the liquid-tight structure 9 can be collected by the material receiving tray 48. Thus, if the amount of the leaked ultrasonic propagation material 6 is small and the leakage thereof occurs temporarily, there is no problem on an operation of the ultrasonic imaging apparatus 52.

With the configuration in FIGS. 16 and 17, it is possible to perform management such that the liquid level of the ultrasonic Propagation material 6 is maintained to be at a position which is suitable with respect to the transducer array 4. It can be detected whether or not the ultrasonic propagation material 6 is leaked from the measurement unit 49 or the hose 7. Thus, it is possible to secure certainty of the measurement result.

FIG. 18 is a diagram illustrating a modification example of the containment vessel. In this modification example, a portion of the side wall of the containment vessel 5 is a stretchable wall 47 which can be stretched. FIG. 18A illustrates a state where the stretchable wall 47 is retracted. FIG. 18B illustrates a state where the stretchable wall 47 is stretched. As described above, the portion of the side wall of the containment vessel 5 is a stretchable wall 47 is configured to be the stretchable wall 47, and thus a configuration in which the lower surface of the containment vessel 5 does not move up and down and the liquid-tight structure 9 is not required is made. The signal wiring 11 is disposed on the side wall other than the stretchable wall 47 or is disposed at the fitting portions 13 and 16. Thus, the signal wiring 11 does not disturb motion of the stretchable wall 47. Other components are similar to those in FIG. 2A. With this configuration, the volume of the containment vessel 5 is changed in accordance with the position of the transducer array 4, and thus the amount of the required ultrasonic propagation material 6 is also changed. However, the liquid-tight structure 9 is not required and the material receiving tray 48 is also not required. The change of the volume of the containment vessel 5 can be solved by setting the volume of the material reservoir 21 to be sufficient.

As a modification example of the measurement unit 49, a wall may be provided between the hole structure 10 and the liquid-tight structure 9, and thus the ultrasonic propagation material 6 may be caused not to directly come into contact with the side wall of the water tank 1. In this case, a third ultrasonic propagation material may be provided between the side wall of the water tank 1 and the measurement unit 49, and acoustic impedance of the paths may match with each other.

According to the above configurations, since the transducer array 4 is installed on the outside of the water tank 1, and the measurement unit 49 which includes the transducer array 4 and the ultrasonic propagation material 6 moves up and down along the side wall of the water tank 1, it is possible to three-dimensionally measure a target. Since the amount of the ultrasonic propagation material 6 in the containment vessel 5 is reduced, maintenance management of the ultrasonic propagation material 6 is easily performed. It is possible to treat the ultrasonic propagation material 6 with high accuracy, and to obtain the shape of a target 3 and acoustic characteristics in a wide range which is close to the vicinity of the opening portion of the water tank 1.

### REFERENCE SIGNS LIST

1 water tank
2, 6 ultrasonic propagation material
3 measurement target
4 transducer array
5 containment vessel
9 liquid-tight structure
10 bubble removal mechanism
12 stage
13, 16 fitting portion
14 driving device
15 guide rail
20, 27 material circulation unit
21 material reservoir
28 electronic control device
31 transmission and reception unit
35 control unit
47 stretchable wall
48 material receiving tray
49 measurement unit
50 main apparatus
51 screw structure
53 wave source
54 virtual wave source

1. An ultrasonic signal processing apparatus comprising:
a water tank which has an opening portion;
a measurement unit which includes a transducer array configured to transmit and receive an ultrasonic signal and a space which is filled with a material for propagating the ultrasonic signal, is disposed on an outside of the water tank, and is configured to measure the ultrasonic signal; and
a moving unit which is connected to the measurement unit and moves the measurement unit between the opening portion and a bottom portion of the water tank, along a side wall of the water tank.

9. The ultrasonic signal processing apparatus according to claim 1, further comprising:
a material circulation unit that circulates the material,
wherein a circulation direction of the material is directed from a lower portion of the measurement unit to an upper portion thereof.

3. The ultrasonic signal processing apparatus according to claim 2,
wherein the measurement unit has a liquid-tight structure between the water tank and a wall of the measurement unit on a lower surface which is a bottom portion side of the water tank, and
the ultrasonic signal processing apparatus further comprises a tray for collecting and receiving the material which is leaked from the liquid-tight structure.

4. The ultrasonic signal processing apparatus according to claim 2, further comprising:
a support unit which is disposed at a position which faces the side wall of the water tank and is configured to support movement of the measurement unit, which is performed by the moving unit.

5. The ultrasonic signal processing apparatus according to claim 1,
wherein a wall of the measurement unit on an upper surface which is the opening portion side of the water tank is thinner than the wall of the measurement unit on a lower surface thereof which is a bottom side of the water tank.

6. The ultrasonic signal processing apparatus according to claim 5,
wherein the wall on the upper surface has a ventilation structure for causing a gas in the measurement unit to pass therethrough.

7. The ultrasonic signal processing apparatus according to claim 6,
wherein the ventilation structure is in contact with the water tank.

8. The ultrasonic signal processing apparatus according to claim 6,
wherein the ventilation structure is a foam structure having elasticity or a brush structure.

9. The ultrasonic signal processing apparatus according to claim 6,
wherein the ventilation structure is a structure into which the material is permeated.

10. An ultrasonic imaging apparatus which transmits and receives an ultrasonic signal to and from a measurement target and captures an image of the measurement target, the apparatus comprising:
an ultrasonic signal processing apparatus that transmits and receives the ultrasonic signal to and from the measurement target and processes the received ultrasonic signal; and
a control device that controls the ultrasonic signal processing apparatus and calculates a shape of the measurement target or acoustic characteristics based on the ultrasonic signal which has been received by the ultrasonic signal processing apparatus,
wherein the ultrasonic signal processing apparatus is the ultrasonic signal processing apparatus according to claim 1, and
the control device calculates a shape of the measurement target or acoustic characteristics by using a correction parameter which reflects a difference of a sound speed of the ultrasonic signal between the water tank, the material in the water tank, and the material in the measurement unit.

11. The ultrasonic imaging apparatus according to claim 10,
wherein the correction parameter includes virtual position coordinates of a transducer constituting the transducer array, a signal response time between a transmission unit configured to transmit the ultrasonic signal and a transmission-side transducer, a signal response time between a reception unit configured to receive the ultrasonic signal and a reception-side transducer, and directivity regarding a signal delayed time in the virtual transducer.

12. The ultrasonic imaging apparatus according to claim 11,
wherein the control device acquires signal intensity distribution of the ultrasonic signal received by the ultrasonic signal processing apparatus, determines whether or not bubbles are provided in the material, based on the signal intensity distribution, and calculates the correction parameter based on a determination result.

13. A control method in an ultrasonic signal processing apparatus which includes a water tank having an opening portion and processes an ultrasonic signal which is transmitted and received to and from an inside of the water tank, the method comprising:
moving a measurement unit which is disposed on an outside of the water tank, includes a transducer array for transmitting and receiving the ultrasonic signal and a space which is filled with a material for propagating the ultrasonic signal, and measures the ultrasonic signal, between the opening portion and a bottom portion of the water tank along a side wall of the water tank.
